(19) United States
(12) Patent Application Publication
(10) Pub. No.: US 2008/0303019 A1
(43) Pub. Date: Dec. 11, 2008

(54) Side Chain-Containing Type Organic Silane Compound, Thin Film Transistor and Method of Producing Thereof

(76) Inventors: Masatoshi Nakagawa, Nara (JP); Hiroyuki Hanato, Nara (JP); Toshihiro Tamura, Nara (JP)

Correspondence Address: NIXON & VANDERHYE, PC, 901 NORTH GLEBE ROAD, 11TH FLOOR, ARLINGTON, VA, 22203, US

(21) Appl. No.: 11/658,168

(22) PCT Filed: Aug. 12, 2005
(86) PCT No.: PCT/JP2005/014847

§ 371 Date: Jan. 23, 2007

(30) Foreign Application Priority Data
Aug. 24, 2004 (JP) 2004-243965
Aug. 24, 2004 (JP) 2004-243974

(51) Int. Cl. H01L 51/05; C07F 7/18; C07D 333/02; C07F 7/08

(52) U.S. Cl. 257/40; 556/482; 549/4; 556/432; 257/E51.006

(57) ABSTRACT

A side chain-containing type organic silane compound represented by the formula (I) R—SiX¹X²X³ wherein R represents a π-electron conjugate type organic residue composed of 3 to 10 units whose units are a group derived from a monocyclic aromatic hydrocarbon, a group derived from a monocyclic heterocyclic compound or the combination thereof, or an organic residue composed of 2 to 10 five-membered or six-membered rings, both of the residues having at least one side chain and X¹, X² and X³, the same or different represent a group affording a hydroxyl group upon hydrolysis.

### TECHNICAL FIELD

The invention relates to a side chain-containing type organic silane compound, an organic thin film transistor and a method of producing thierof, and, particularly, to a side chain-containing type organic silane compound which is a conductive or semiconductive novel material useful as electronic materials, an organic thin film transistor and a method of producing thierof.

### BACKGROUND ART

Besides semiconductors using inorganic materials, semiconductors (organic semiconductors) using organic compounds have been recently researched and developed and the results have been reported because these organic semiconductors are simply produced and easily processed, can correspond to the miniaturization of devices and is expected to attain cost reduction in mass-production, and as the organic compounds, various organic compounds having a more variety of functions than inorganic materials can be synthesized.

It is known that TFTs having large mobility can be produced by utilizing organic compounds containing a π-electron conjugate molecule among these organic materials. As this organic compound, pentacene is reported as a typical example (for example, IEEE Electron Device Lett., 18, 606-608 (1997): Non-patent Document 1). In this report, there is the description that when pentacene is used to produce a semiconductor layer, which is used to form a TFT, the field effect mobility is 1.5 cm²/Vs and it is therefore possible to produce a TFT having a larger mobility than amorphous silicon.

However, when an organic semiconductor layer having a higher field effect mobility than amorphous silicon as shown above is produced, a vacuum process such as a resistance heating vapor deposition method and a molecular beam vapor deposition method is required. This leads to the result that the production process is complicated and a crystalline film is obtained only under a specific condition. Also, this method has the problem that because the adsorption of the organic compound film to the substrate in the vacuum process is physical adsorption and therefore, the adsorption strength of the film to the substrate is so low that the film is easily peeled off. Generally, the orientation of a substrate on which the film is to be formed is controlled by rubbing treatment or the like to control the orientation of the molecules of the organic compound in the film to some extent. However, there has been no report concerning the fact that the conformity and orientation of a compound molecule at the boundary between the physically adsorbed organic compound film and the substrate can be controlled by the film formation by physical adsorption yet.

On the other hand, studies as to the orderliness (regularity and crystallinity) of a film which have a large influence on the field effect mobility that is a typical guide to the characteristics of a TFT. Recently, a self-organizing film using an organic compound that is simply produced is attracting attention, and a use of the self-organizing film is studied.

The self-organizing film means a film which is obtained by combining a part of an organic compound with a functional group present on the surface of a substrate, is very reduced in defects and has high orderliness, that is, high crystallinity. This self-organizing film is formed on the substrate with ease because it is produced by a very simple production method. Generally, a thiol film formed on a gold substrate and a silicon type compound film formed on a substrate (for example, a silicon substrate) are known as the self-organizing film, and the later substrate can be processed by hydrophilic treatment such that a hydroxyl group is allowed to project from its surface. Among these films, a silicon type compound film attracts remarkable attention from the viewpoint of high durability. The silicon type compound film is conventionally used as a water-repellent coating and is formed using a silane coupling agent containing, as organic functional groups, an alkyl group or fluorinated alkyl group having a high water-repellent effect.

However, the conductivity of the self-organizing film is determined by an organic functional group in a silicon type compound contained in the film. However, no commercially available silane coupling agent is found which contains a π-electron conjugate molecule as an organic functional group. It is therefore difficult to impart conductivity to the self-organizing film. There is therefore a strong demand for a silicon compound which is suitable to a device such as a TFT and contains a π-electron conjugate molecule as an organic functional group.

As such a silicon type compound, a compound is proposed which has one thiophene ring as a functional group on the terminal of a molecule, the thiophene ring being connected with a silicon atom through a straight-chain hydrocarbon group (for example, Japanese Patent No. 2889768: Patent Document 1).

[Non-patent Document 1] IEEE Electron Device Lett., 18, 606-608 (1997)

[Patent Document 1] Japanese Patent No. 2889768

### DISCLOSURE OF INVENTION

### Problems that the Invention is to Solve

The compound proposed above ensures the production of a self-organizing film that can be chemically adsorbed to a substrate. However, it has unnecessarily ensured the production of a thin film having high orderliness, crystallinity and electroconductive characteristics enough to produce electronic devices such as TFTs.

In order to obtain high orderliness, that is, high crystallinity, it is necessary that high attracting interaction is exerted between molecules. The intermolecular force is constituted of an attractive factor and a repulsive factor, wherein the former is in inverse proportion to the 6th power of the distance between molecules and the latter is in inverse proportion to the 12th power of the distance between molecules. Therefore, the intermolecular force which is the sum of the attractive factor and the repulsive factor has the relationship as shown in FIG. 1. Here, the minimum point (the point indicated by the arrow in the figure) indicates the distance between molecules at which the highest attractive force is exerted between molecules in the balance between the attractive factor and the repulsive factor. Specifically, it is important that the intermolecular distance is made to be the closest to the minimum point to obtain high crystallinity. Therefore, originally, in a vacuum process such as a resistance heating vapor deposition method and a molecular beam vapor deposition method, a film having high orderliness, specifically, high crystallinity is obtained by well controlling the intermolecular interaction between π-electron conjugate molecules only in a certain specific condition. It is possible to develop high electroconductive characteristics only when the film has such the high crystallinity structured by intermolecular interaction.

On the other hand, the above compound has the possibility of forming a two-dimensional network of Si—O—Si so that it is chemically adsorbed to the substrate and also, the orderliness by the intermolecular interaction between specific long-chain alkyls is obtained. However, this compound has the problem that it has low intermolecular interaction because only one thiophene molecule that is a functional group contributes to a π-electron conjugate system and a spread of the π-electron conjugate system which is essential for electroconductivity is very small. Even if the number of thiophene molecules which are the above functional groups could be increased, it is difficult that the factors forming the orderliness of the film are coordinately and consistent with the intermolecular interaction between the long-chain alkyl part and the thiophene part.

As to the electroconductive characteristics, only one thiophene molecule which is a function group has a large HOMO-LUMO energy gap, giving rise to the problem that only insufficient carrier mobility is obtained even if a TFT is used in an organic semiconductor layer.

The present invention has been made in view of the above problem and has the following object. Specifically, it is an object of the present invention to provide a compound which can be easily crystallized by a simple production method using a solution process to form a thin film, makes the obtained thin film adsorb to the surface of a substrate firmly to prevent the thin film from being peeled off physically and has high orderliness, crystallinity and electroconductivity. Further, it is an object of the present invention to provide a novel organic silane compound which can secure sufficient carrier mobility when used as an electronic device such as a TFT and a method of producing the compound.

### Means of Solving the Problems

The inventors of the present invention have made earnest studies and as a result, found that an organic silane compound capable of forming an organic thin film adaptable to electronic devices such as TFTs must have:

(1) a structure capable of forming a two-dimensional network of Si—O—Si which can be chemically bonded to a substrate firmly; and

(2) a structure enabling the orderliness (crystallinity) of an organic thin film to be controlled by the interaction, that is, intermolecular force, between molecules (π-electron conjugate type molecules) on a two-dimensional network of Si—O—Si. Thus, the inventors have invented a novel organic silane compound having these structures.

Accordingly, the present invention provides a side chain-containing type organic silane compound represented by the formula (I) R—SiX¹X²X³ wherein R represents a π-electron conjugate type organic residue composed of 3 to 10 units whose units are a group derived from a monocyclic aromatic hydrocarbon, a group derived from a monocyclic heterocyclic compound or the combination thereof, or an organic residue composed of 2 to 10 five-membered or six-membered rings, both of the residues having at least one side chain and X¹, X² and X³, the same or different represent a group affording a hydroxyl group upon hydrolysis.

According to the present invention, there is provided a method of producing a side chain-containing type organic silane compound comprising reacting a compound represented by the formula (II) R—Li, wherein R represents a π-electron conjugate type organic residue composed of 3 to 10 units whose units are a group derived from a monocyclic aromatic hydrocarbon, a group derived from a monocyclic heterocyclic compound or the combination thereof, or an organic residue composed of 2 to 10 five-membered or six-membered rings, both of the residues having at least one side chain,

with a compound represented by the formula (III) Y—SiX¹X²X³, wherein X¹, X² and X³, the same or different represent a group affording a hydroxyl group upon hydrolysis and Y represents a hydrogen atom, a halogen atom or a lower alkoxy group

to produce the side chain-containing type organic silane compound represented by the formula (I) R—SiX¹X¹X²X³, wherein R and X1 to X3 are as defined above.

According to the present invention, there is provided a method of producing a side chain-containing type organic silane compound comprising reacting a compound represented by the formula (IV) R—MgX, wherein R represents a π-electron conjugate type organic residue composed of 3 to 10 units whose units are a group derived from a monocyclic aromatic hydrocarbon, a group derived from a monocyclic heterocyclic compound or the combination thereof, or an organic residue composed of 2 to 10 five-membered or six-membered rings, both of the residues having at least one side chain,

with a compound represented by the formula (III) Y—SiX¹X²X³, wherein X¹, X² and X³, the same or different represent a group affording a hydroxyl group upon hydrolysis and Y represents a hydrogen atom, a halogen atom or a lower alkoxy group

to produce the side chain-containing type organic silane compound represented by the formula (I) R—SiX¹X¹X²X³, wherein R and X1 to X3 are as defined above.

Further, the present invention provides an organic thin film transistor comprising a substrate, an organic thin film, a gate electrode formed on one surface of the organic thin film through a gate insulating film and a source/drain electrode formed in contact with one surface or the other surface of the above organic thin film on both sides of the gate electrode, wherein the above organic thin film is a film derived from a side chain-containing type organic silane compound represented by the formula (I) R—SiX¹X²X³ wherein R represents a π-electron conjugate type organic residue composed of 3 to 10 units whose units are a group derived from a monocyclic aromatic hydrocarbon, a group derived from a monocyclic heterocyclic compound or the combination thereof, or an organic residue composed of 2 to 10 five-membered or six-membered rings, both of the residues having at least one side chain and X¹, X² and X³, the same or different represent a group affording a hydroxyl group upon hydrolysis.

Further, the present invention provides a process of producing an organic thin film transistor comprising a substrate, an organic thin film, a gate electrode formed on one surface of the organic thin film through a gate insulating film and a source/drain electrode formed in contact with one surface or the other surface of the above organic thin film on both sides of the gate electrode, the process comprising a step of forming an organic thin film by laminating, as a monomolecular film or built-up film, a side chain-containing type organic silane compound represented by the formula (I) R—SiX¹X²X³ wherein R represents a π-electron conjugate type organic residue composed of 3 to 10 units whose units are a group derived from a monocyclic aromatic hydrocarbon, a group derived from a monocyclic heterocyclic compound or the combination thereof, or an organic residue composed of 2 to 10 five-membered or six-membered rings, both of the residues having at least one side chain and X¹, X² and X³, the same or different represent a group affording a hydroxyl group upon hydrolysis.

### EFFECT OF THE INVENTION

Since the compound of the present invention contains a side chain in the organic residue, it is therefore highly soluble in an organic solvent and can easily form a film by a solution type process. The compound of the present invention can be therefore chemically bonded to the substrate due to a network structure which is constituted of silicon atoms and oxygen atoms and formed between neighboring compounds by silyl groups contained in the organic silane compound. In addition, because the intermolecular force interacted among π-electron conjugate molecules works effectively and it is therefore expected that an organic thin film which has very high stability and is highly crystallized can be constituted.

Also, the compound of the present invention is expected to have high crystallinity when it is formed into a film because intermolecular force works not only between primary chains of the organic residue but also between side chains of the organic residue.

Moreover, the compound of the present invention can provide two different conductivities, namely, particularly high conductivity in a direction perpendicular to the molecular plane of the principal chain of the organic residue and conductivity in another direction. Therefore, the compound of the present invention is expected to be widely applied as conductive material not only to organic thin film transistor materials but also to solar cells, fuel cells, and sensors.

### BRIEF DESCRIPTION OF DRAWINGS

FIG. 1 is a view for explaining the relationship between intermolecular distance and intermolecular force.

FIG. 2 is an outline view of one organic TFT of the present invention.

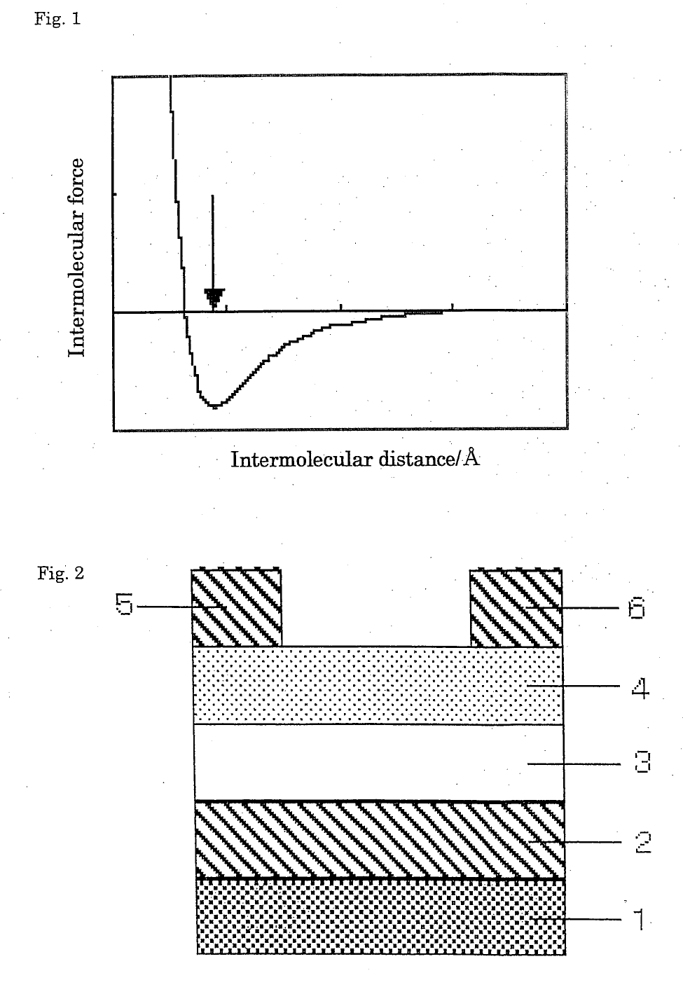

### BEST MODE FOR CARRYING OUT THE INVENTION

The side chain-containing type organic silane compound (hereinafter called simply as “silane compound”) of the present invention is represented by the formula (I), specifically, R—SiX¹X¹X²X³. In the formula (I), R represents a π-electron conjugate type organic residue composed of 3 to 10 units whose units are a group derived from a monocyclic aromatic hydrocarbon, a group derived from a monocyclic heterocyclic compound or the combination thereof, or an organic residue composed of 2 to 10 five-membered or six-membered rings, both of the residues having at least one side chain.

Examples of the monocyclic aromatic hydrocarbons include benzene, toluene, xylene, mesitylene, cumene, cymene, styrene and divinylbenzene. Among these compounds, benzene is preferable.

Examples of the heteroatom contained in the monocyclic heterocyclic compound include oxygen, nitrogen and sulfur. Specific examples of the heterocyclic compound include oxygen atom-containing compounds such as furan, nitrogen atom-containing compounds such as pyrrole, pyridine, pyrimidine, pyrroline, imidazoline and pyrazoline, sulfur atom-containing compounds such as thiophene, nitrogen and oxygen atoms-containing compounds such as oxazole and isoxazole and sulfur and nitrogen atoms-containing compounds such as thiazole and isothiazole. Among these compounds, thiophene is preferable.

Three to ten units among the above units are combined with each other to make the π-electron conjugate type organic residue. Three to eight units among the above units are preferably combined with each other in consideration of yield, economy and mass-production.

Although two or more of these units may be combined branched-wise, they are preferably combined linearly. Also, the organic residue may have a structure in which the same units are combined, all different units are combined or plural types of units are combined regularly or at random. Also, when the unit is a group made of a five-membered ring, the binding positions may be any of 2,5-positions, 3,4-positions, 2,3-positions and 2,4-positions. Among these positions, 2,5-positions are preferable. In the case of six-membered ring, the binding positions may be any of 1,4-positions, 1,2-positions and 1,3-positions. Among these combinations, 1,4-positions are preferable.

Moreover, a vinylene group may be positioned between these units. Examples of the hydrocarbon providing the vinylene group include alkenes, alkadienes and alkatrienes. Examples of the alkenes include compounds having 2 to 4 carbon atoms such as ethylene, propylene and butylene. Among these compounds, ethylene is preferable. Examples of the alkadiene include compounds having 4 to 6 carbon atoms such as butadiene, pentadiene and hexadiene. Examples of the alkatriene include compounds having 6 to 8 carbon atoms such as hexatriene, heptatriene and octatriene.

Specific examples of R include groups derived from a biphenyl, bithiophenyl, terphenyl (compound of the formula (1)), terthienyl (compound of the formula (2)), quarter phenyl, quarter thiophene, quinque-phenyl, quinque-thiophene, hexyphenyl, hexythiophene, thienyl-oligophenylene (compounds of the formula (3)), phenyl-oligooligothienylene (compounds of the formula (4) block oligomer (for example, compounds of the formula (5) or (6)) and bi(dithiophenylvinyl)phenyl (compound of the formula (7)).

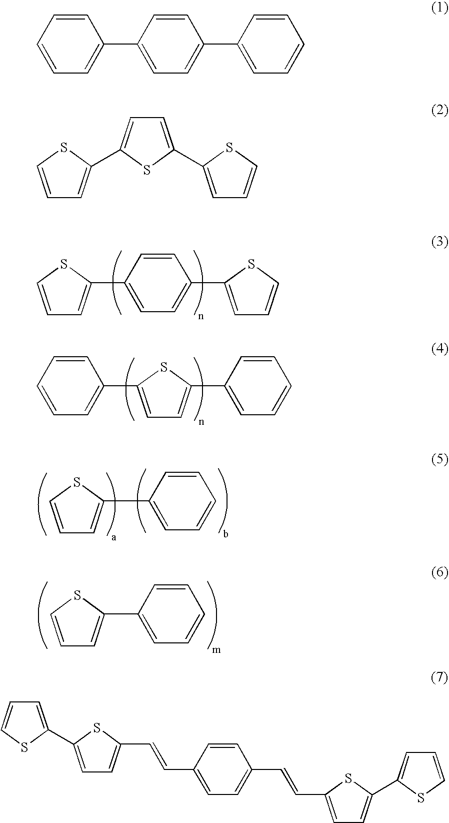

Here, R may have linear symmetry with respect to the molecular axis or point symmetry with respect to the center as a whole. For example, in the above formula, (1) has line symmetry and (2) to (4) have point symmetry.

As the condensed polycyclic compound, any compound may be used insofar as it has aπ-electron conjugate type molecular structure and those having symmetry and particularly, line symmetry are preferable from the viewpoint of conductivity. Also, condensed polycyclic compounds consisting of 2 to 10 condensed five-membered or six-membered rings are preferable in view of productivity. Specific examples of the skeleton of such a preferable compound include an acene skeleton which is a linear condensed cyclic type, aphene skeleton which is a wing condensed cyclic type, arene skeleton which is a condensed cyclic type in which the same two rings are lined up and acene skeleton in which condensed type benzene rings center at one ring or phenylene skeleton. Among these skeletons, particularly the acene skeleton or phenylene skeleton in which benzene rings are bounds linearly are preferable in consideration of carrier mobility. Specific examples of the acene skeleton include naphthalene, anthracene, tetracene (naphthacene), pentacene, hexacene, heptacene and octacene. Examples of the phenylene skeleton include phenalene, perylene, coronene and ovalene. Among these skeletons, acene skeletons in which the number of benzene rings is 2 to 10 (n=0 to 8) as shown by the following structure are preferable.

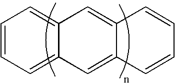

Moreover, in the case where R is a condensed polycyclic compound, R may be bound with Si through the aryl group R2 bound with the organic residue. Examples of the aryl group include a phenyl group derived from benzene or biphenyl, a thienyl group derived from thiophene or bithiophene and a group constituted of a combination of these groups wherein a vinylene group may be contained between the groups.

The above organic residue may have a functional group at its terminal. Specific examples of the functional group include a hydroxyl group, substituted or unsubstituted amino group, nitro group, cyano group, substituted or unsubstituted alkyl group, substituted or unsubstituted alkenyl group, substituted or unsubstituted cycloalkyl group, substituted or unsubstituted alkoxy group, substituted or unsubstituted aromatic heterocyclic group or unsubstituted aralkyl group, substituted or unsubstituted aryloxy group, substituted or unsubstituted alkoxycarbonyl group or a carboxyl group, ester group and trialkoxysilyl group.

No particular limitation is imposed on the group affording a hydroxyl group upon hydrolysis in X¹, X² and X³ involved in the above formula (I) and examples of the group include halogen atoms and lower alkoxy groups. Examples of the halogen atom include fluorine, chlorine, iodine and bromine atoms. Examples of the lower alkoxy group include alkoxy groups having 1 to 4 carbon atoms. Examples of these alkoxy groups include a methoxy group, ethoxy group, n-propoxy group, 2-propoxy group, n-butoxy group, sec-butoxy group and tert-butoxy group wherein a part of each of these groups may be further substituted with other functional groups (trialkylsilyl group or other alkoxy groups). X¹, X² and X³, though they may be the same or different, need not all the same. Specifically, two or all of X¹, X² and X³ may be different from each other. However, all of X¹, X² and X³ are preferably the same.

Also, Rs in the above formula (I) respectively have a side chain. Here, the side chain is preferably a group providing the lipophilic ability to improve solubility in an organic solvent. Particularly, the side chain is preferably a group which does not react with neighboring molecules. Examples of the side chain include a substituted or unsubstituted alkyl group, halogenated alkyl group, cycloalkyl group, aryl group, diarylamino group, di or triarylalkyl group, alkoxy group, oxyaryl group, nitrile group, nitro group, ester group, trialkylsilyl group, triarylsilyl group, phenyl group and acene group. Here, the volume occupied by side chain molecules is preferably 100% or less and more preferably 60% or less of the volume occupied by the main skeleton of the organic residue excluding the side chains with the view of promoting the intermolecular interaction between neighboring molecules and improving the crystallinity of the organic thin film to thereby provide high conductivity in consideration of using as organic thin film materials. This is because if the molecular occupying volume exceeds 100% of that of the main skeleton, the intermolecular interaction among the principal skeletons is smaller than that of the side chains and there is therefore the case where the crystallinity is significantly reduced.

As such a side chain, a linear alkyl group having 1 to 4 carbon atoms, di or trialkylsilyl group having 1 to 4 carbon atoms, branched alkyl group in which an alkyl group having 1 to 4 carbon atoms is bound with a secondary or tertiary hydrocarbon, mono, di or triarylalkyl group containing an aryl group having 5 to 18 carbon atoms, mono, di or triarylsilyl group containing an aryl group having 5 to 18 carbon atoms or di or triarylamino group containing an aryl group having 5 to 18 carbon atoms is preferable. Particularly, an aryl group (for example, a phenyl group or a group derived from naphthalene and anthracene) having 1 to 3 benzene rings, tertiary alkyl group containing an alkyl group having 1 to 4 carbon atoms and triarylalkyl or triarylsilyl group containing an aryl group (for example, a phenyl group or a group derived from naphthalene or anthracene) having 1 to 3 benzene rings are preferable. Specific examples of the silane compound according to the present invention include those shown below.

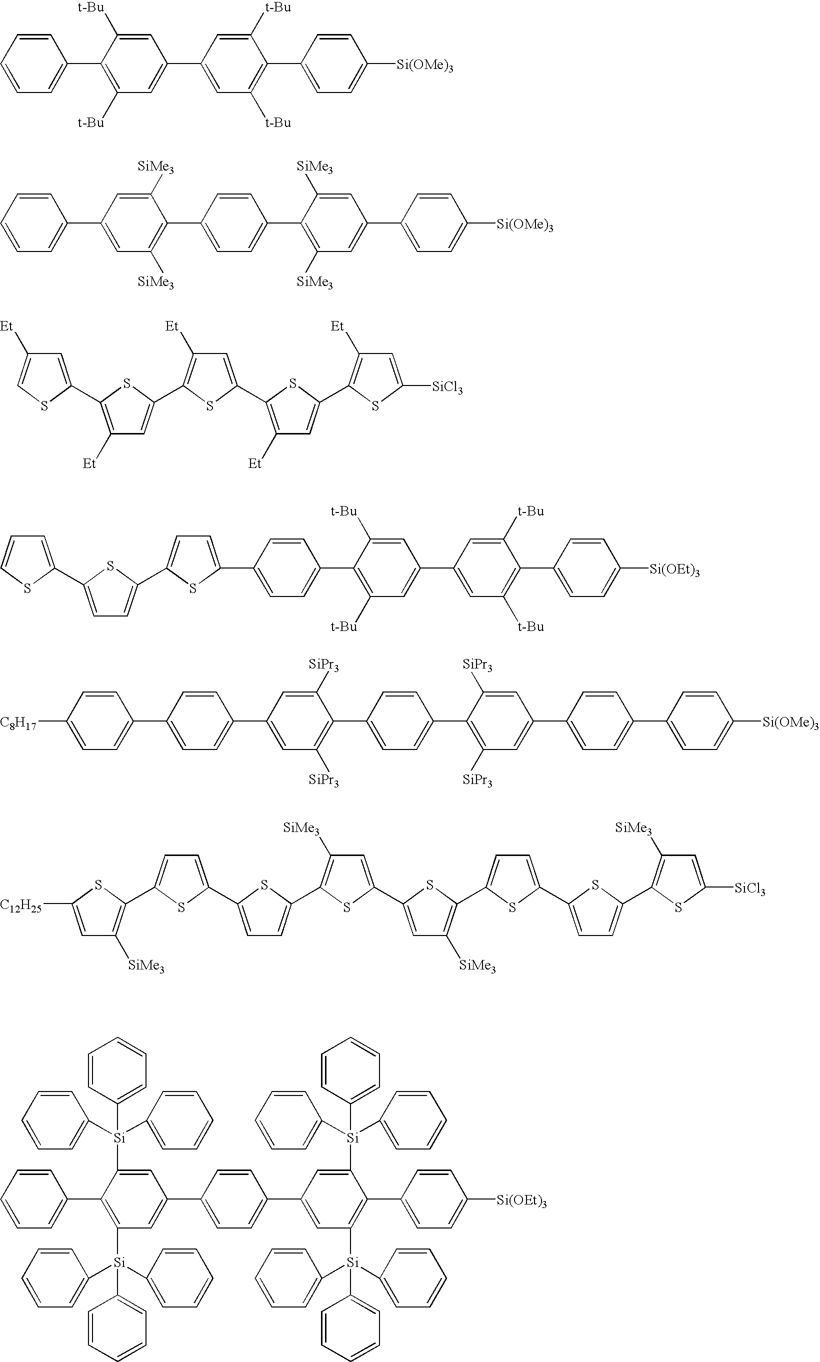

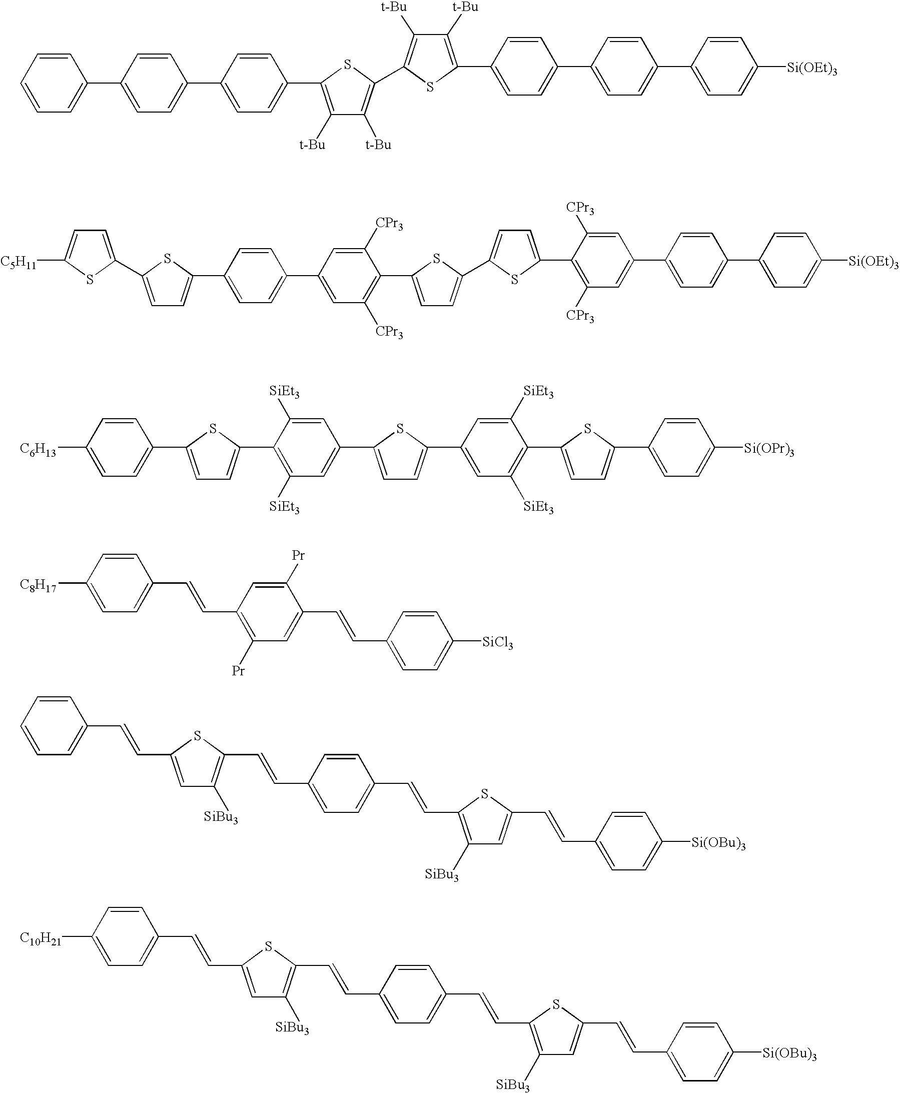

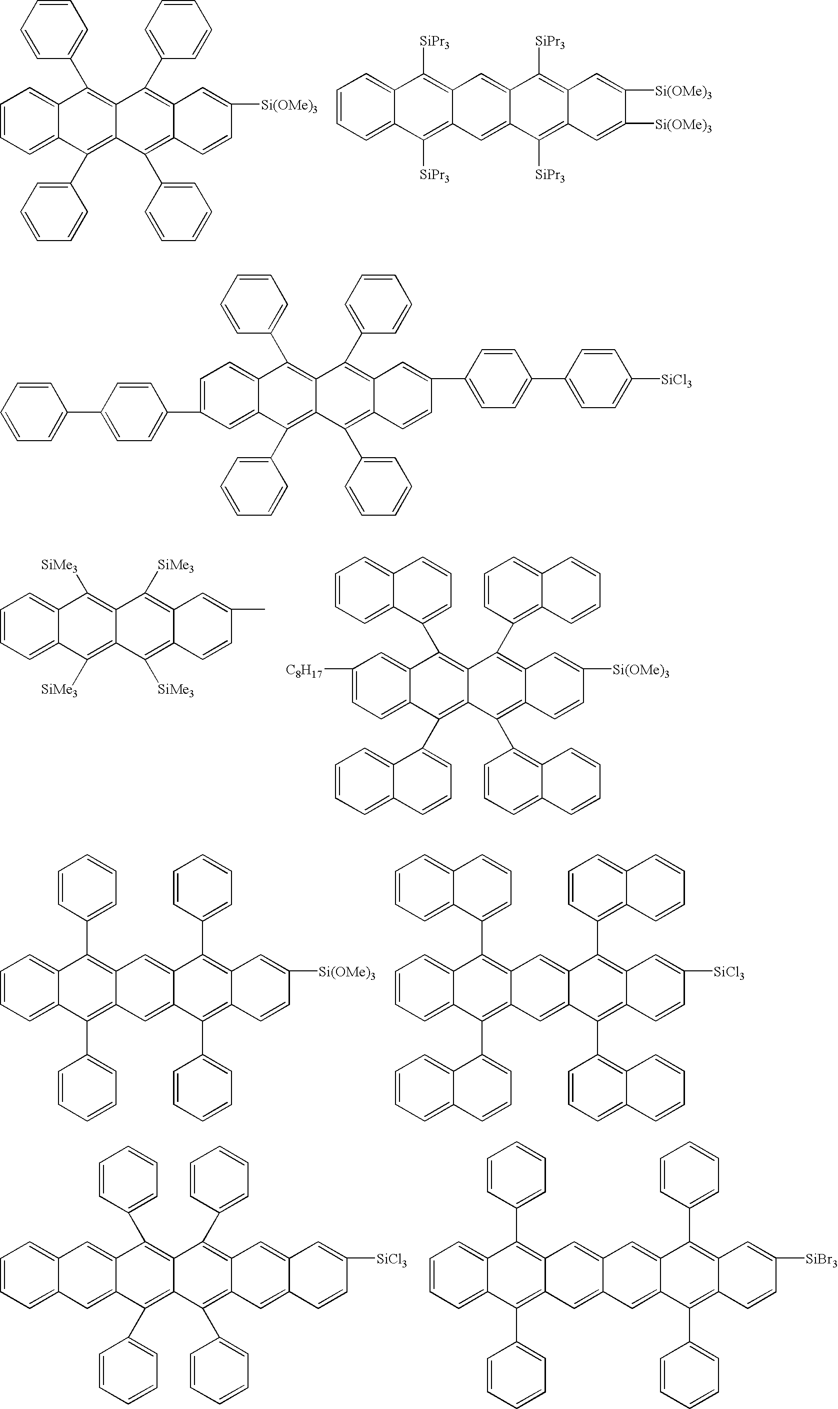

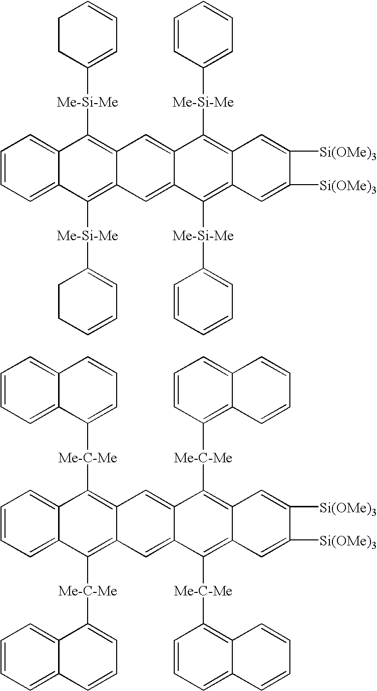

A process of synthesizing the silane compound of the present invention will be explained.

The silane compound of the present invention may be obtained by;

reacting a compound represented by the formula R—Li (II) with a compound represented by the formula Y—SiX¹X²X³ (III) (wherein X¹, X² and X³ and Y have the same meanings as those defined above); or

running a Grignard's reaction between a compound represented by the formula R—MgX (wherein R and X have the same meanings as above) with a compound represented by the above formula (III).

The compound represented by the formula (II) or (IV) may be obtained by reacting a compound represented by RH with an alkyl lithium or by reacting a compound represented by the formula R—X (X is a halogen atom) with an alkyl magnesium halide or metal magnesium or the like.

The temperatures in the reaction of compounds (II) and (III) and the reaction of compounds (IV) and (III) are, for example, −100 to 150° C. and preferably −20 to 100° C. The reaction times are, for example, about 0.1 to 48 hours. The reactions are usually run in an organic solvent having no influence on the reaction. Examples of the organic solvent having no influence on the reactions include hydrocarbons such as hexane, pentane, benzene and toluene, ether type solvents such as diethyl ether, dipropyl ether, dioxane and tetrahydrofuran (THF) and aromatic hydrocarbons such as benzene and toluene. These organic solvents may be used either singly or as a mixture solution. Among these solvents, diethyl ether and THF are preferable. In the reaction, a catalyst may be optionally used. As the catalyst, a known catalyst such as a platinum catalyst, palladium catalyst or nickel catalyst may be used.

The method of synthesizing a silane compound according to the present invention will be explained. The reaction temperature and the reaction time in the following synthetic methods are the same as those mentioned above and are, for example, −100 to 150° C. and 0.1 to 48 hours.

The following explanations are furnished as to a synthetic example of a precursor of the organic residue group constituted of a unit derived from benzene which is an example of the monocyclic aromatic hydrocarbon and a unit derived from thiophene which is an example of the monocyclic heterocyclic compound. A precursor of heterocyclic compounds containing a nitrogen atom or an oxygen atom may also be produced in the same manner as in the production of a nitrogen-containing heterocyclic compound such as thiophene. Though the side chain is not described below to avoid complexity, the side chain can be introduced by using a raw material having a halogen atom at a desired position and utilizing a Grignard's reagent.

As a method of synthesizing the precursor constituted of a unit derived from benzene or thiophene, a method in which, first, the reaction part of benzene or thiophene is halogenated and then, a Grignard reaction is utilized is effective. The use of this method makes it possible to synthesize a precursor in which the number of benzenes or thiophenes is controlled. Besides the method in which a Grignard's reagent is used, the precursor may be synthesized by coupling utilizing a proper metal catalyst (Cu, Al, Zn, Zr or Sn, etc.).

As to thiophene, the following synthetic methods may be utilized, as well as the method to which a Grignard's reagent is applied.

Specifically, first, the 2-position or 5-position of thiophene is halogenated (for example, brominated chlorinated). Examples of the halogenating method include one-equivalent N-chlorosuccinimide (NCS) treatment and phosphorous oxychloride (POCl₃) treatment. As the solvent at this time, for example, a chloroform/acetic acid (AcOH) mixture solution or DMF may be used. Also, halogenated thiophenes are reacted among them under the presence of tris(triphenylphosphine)nickel (PPh)₃Ni as a catalyst in a DMF solvent, whereby these thiophenes can be combined at the halogenated parts resultantly.

Moreover, divinylsulfone is added to the halogenated thiophene to couple the both, thereby forming a 1,4-diketone body. In succession, a Lawesson Reagent (LR) or P₄S₁₀ is added to the 1,4-diketone body and the resulting mixture is refluxed overnight in the former case or for 3 hours in the latter case to cause a ring-closing reaction. As a result, a precursor having the number of thiophenes larger by one than the total number of the coupled thiophenes can be synthesized.

The number of thiophene rings can be increased by utilizing the above reaction of thiophene.

The above precursor may be halogenated at its terminal in the same manner as in the case of the raw material used for the synthesis. Therefore, the precursor is halogenated and then, reacted with, for example, SiCl₄ to obtain a silicon compound (simple benzene or simple thiophene compound) which has a silyl group at its terminal and is provided with an organic group (R1) constituted only of a unit derived from benzene or thiophene.

One example of a method of synthesizing the precursor of the organic group constituted only of benzene or thiophene and one example of a method of silylating the precursor are shown in the following (A) to (D). In this case, in the synthetic example of the precursor constituted only of thiophene, only reactions of thiophene trimers into hexamers or heptamers are shown. However, if this thiophene is reacted with a thiophene having different unit number, precursors other than the above hexamers or heptamers can be formed. For example, if 2-chlorobithiophene chlorinated by NCS after 2-chlorothiophene is coupled is reacted in the same manner as in the following method, a thiophene tetramer or pentamer can be formed. Moreover, if the thiophene tetramer is chlorinated by NCS, a thiophene octamer or nonamer can also be formed.

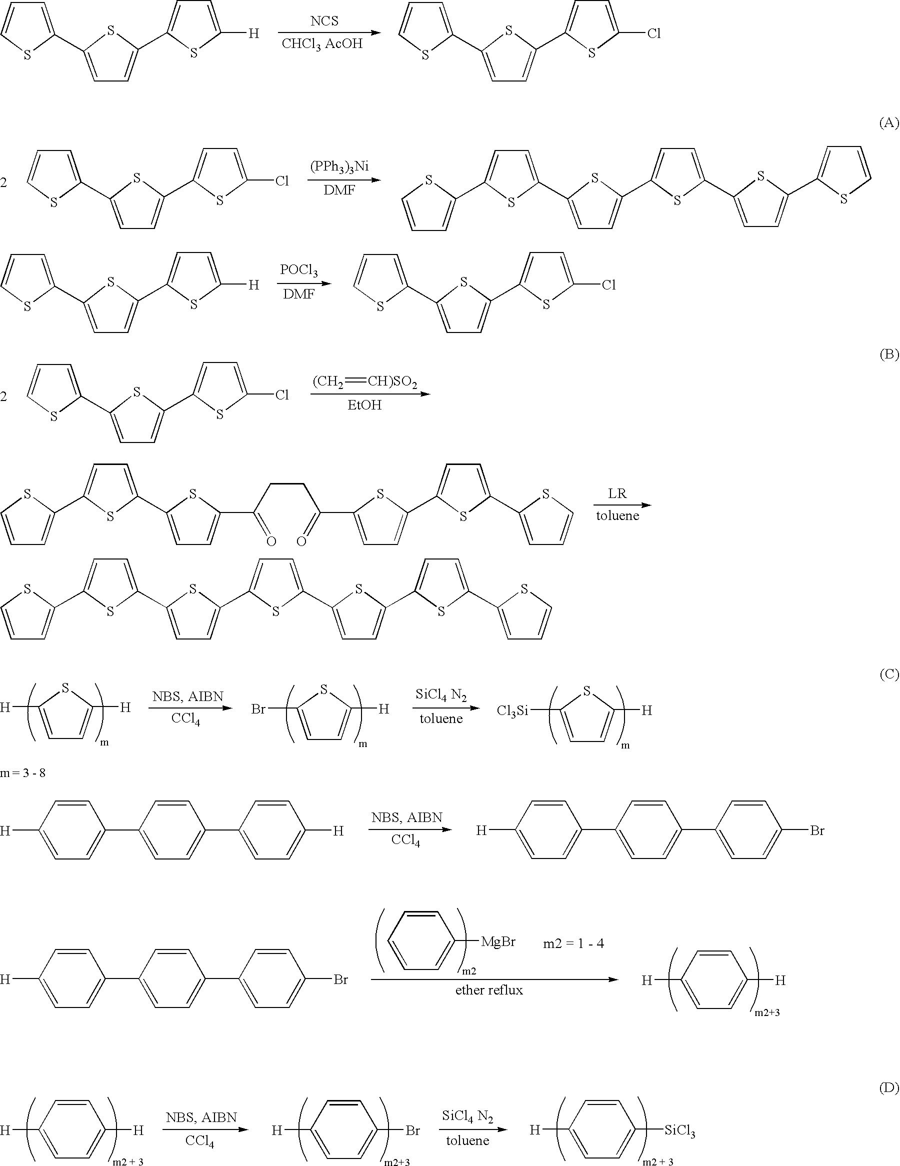

There is, for example, a method using a Grignard reaction as a method used to obtain a block type organic group precursor by directly binding units obtained by binding units derived from a specified number of thiophenes or benzenes. If the precursor is reacted with SiCl₄ or HSi(OEt)₃, a target silicon compound can be obtained. Also, among the above compounds, the compound having a terminal alkoxy group and a silyl group can be synthesized in the condition that it is bound with the raw material in advance because it has low reactivity. As synthetic examples in this case, the following method may be applied.

First, an opposite terminal of a silyl group of a simple benzene or simple thiophene compound is halogenated (for example, brominated) and then, the functional group combined with the silyl group is converted from the halogen into an alkoxy group by a Grignard reaction. In succession, n-BuLi and B(O-iPr)₃ are added to carry out debromination and the formation a boron compound. The solvent used at this time is preferably an ether. Also, the reaction when the boron compound is formed is preferably run in two stages: the reaction is run at −78° C. in the first stage to stabilize the reaction in the initial stage and at temperatures raised gradually from −78° C. to ambient temperature in the second stage. In the meantime, an intermediate of a block type compound is produced by a Grignard reaction using benzene or thiophene having halogen groups (for example, a bromo group) at both terminals.

In this state, if the intermediate having unreacted bromo group and the above boron compound are placed in, for example, a toluene solvent and are reacted completely at a reaction temperature of 85° C. in the presence of Pd(PPh₃)₄ and Na₂CO₃, it is possible to cause coupling. As a result, a silicon compound having a silyl group at the terminal of a block type compound can be synthesized.

One example of the synthetic routes of silicon compounds (E) and (F) by using such a reaction is shown below. Here, the compound having a halogen group (for example, a bromo group) and a trichlorosilyl group at both terminals of the unit derived from benzene or thiophene may be formed by reacting p-phenylene or 2,5-thiophenediyl with a halogenating agent (for example, NBS) to halogenate both terminals and then by reacting the reaction product with SiCl₄ to substitute one of the terminal halogen with a trichlorosilyl group.

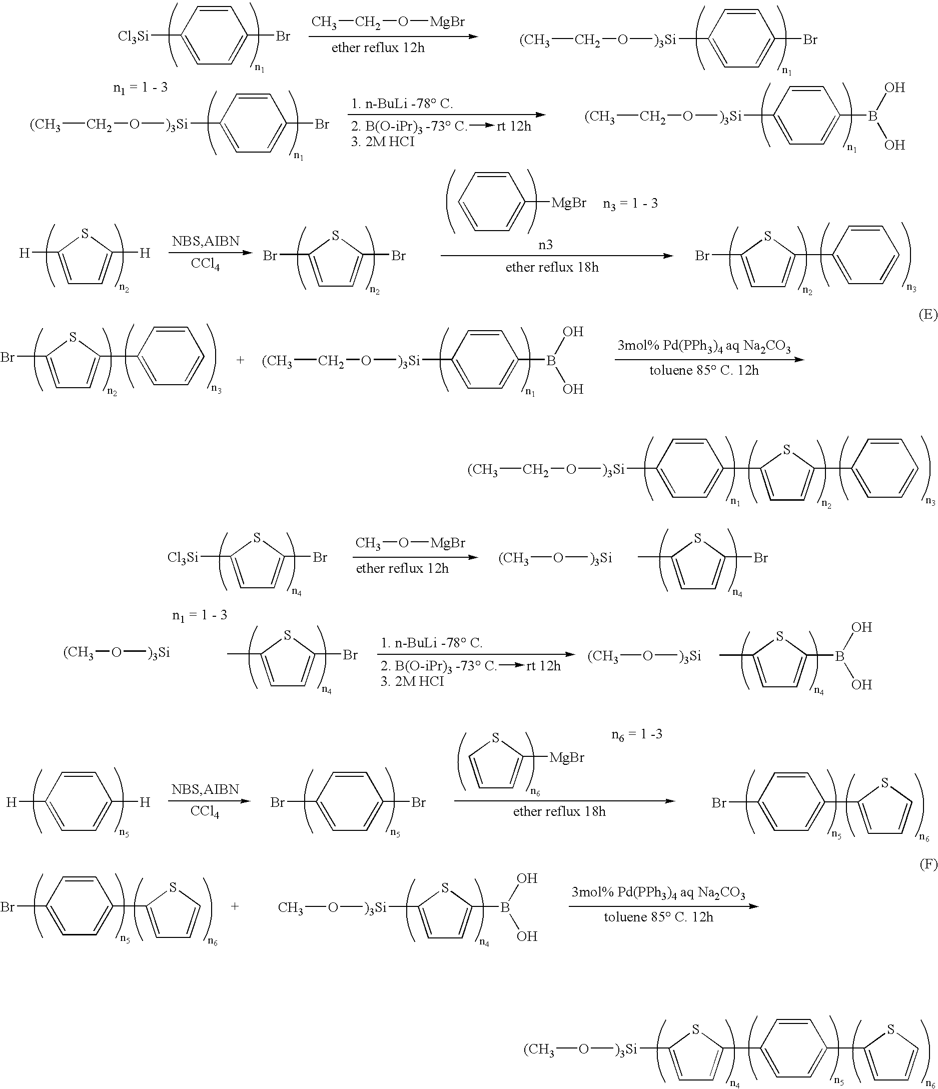

As a method of synthesizing a precursor in which units derived from benzene or thiophene and vinyl groups are alternately bound, for example, the following method may be applied. Specifically, a raw material made of benzene or thiophene provided with a methyl group at its reaction position is prepared and then, its both terminals are brominated by using 2,2′-azobisisobutyronitrile (AIBN) and N-bromosuccinimide (NBS). Thereafter, PO(OEt)₃ is reacted with the bromo body to form an intermediate. In succession, a compound having an aldehyde group at its terminal is reacted with the intermediate in, for example, a DMF solvent by using NaH, whereby the above precursor can be formed. The resulting precursor has a methyl group at its terminal. Therefore, if the methyl group is further brominated and the above synthetic route is applied again, a precursor more increased in the number of units can be formed.

If the obtained precursor is brominated using, for example, NBS, the brominated part can be reacted with SiCl₄. Therefore, a silicon compound having SiCl₃ at its terminal can be formed. One example of the synthetic routes of precursors (G) to (I) differing in length and silicon compound (J) is shown below by the above reaction.

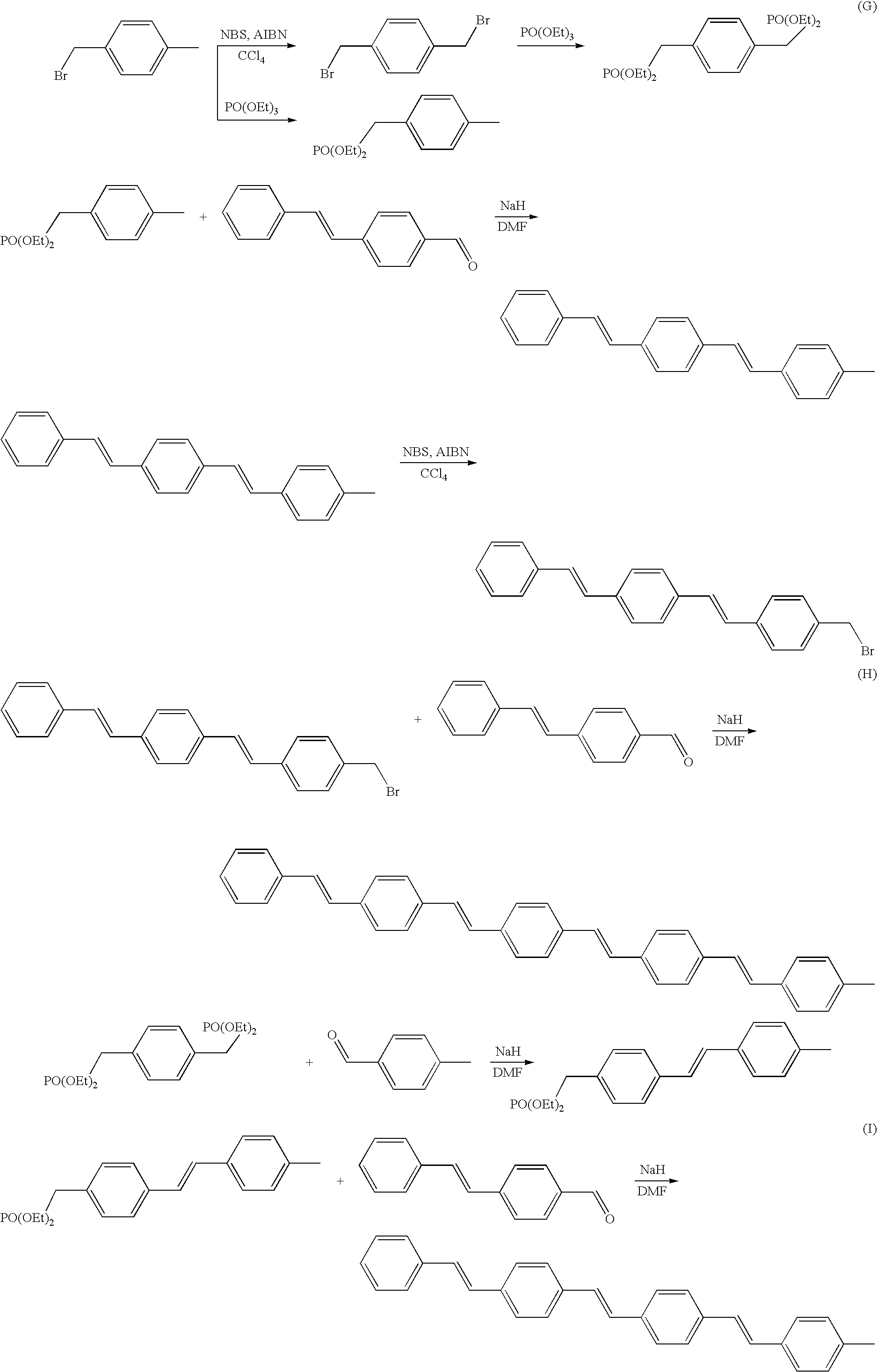

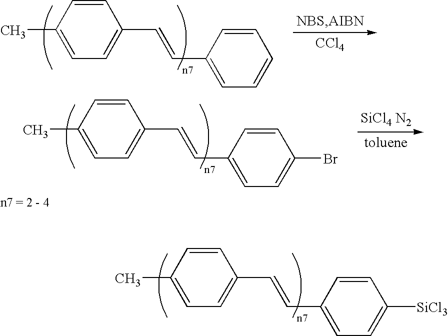

Also, the raw material used in the above synthetic example is a common reagent, which is commercially available from a reagent maker and can be utilized. The CAS number and the purity of a reagent in the case where the reagent maker is Kishida Kagaku are shown below.

TABLE 1

| Raw material | CAS No. | Purity |
|---|---|---|
| 2-chlorothiophene | 96-43-5 | 98% |
| 2,2′,5′,2″-terthiophene | 1081-34-1 | 99% |
| Bromobenzene | 108-86-1 | 98% |
| 1,4-dibromobenzene | 106-37-6 | 97% |
| 4-bromobiphenyl | 92-66-0 | 99% |
| 4,4′-dibromobiphenyl | 93-86-4 | 99% |
| p-terphenyl | 92-94-4 | 99% |
| α-bromo-p-xylene | 104-81-4 | 98% |

Next, a synthetic example of a precursor of the organic residue constituted of a unit derived from an acene skeleton that is an example of the condensed ring constituted of a five-membered or six-membered ring is shown below. In this case, the side chain can be introduced into a desired position of the acene skeleton by using a raw material having a halogen atom at a desired position of R and by utilizing a Grignard's reagent. These synthetic methods are typical examples and other known synthetic methods may be applied.

Examples of a synthetic method of the acene skeleton include (1) a method in which a step of substituting ethynyl groups for hydrogen atoms bound with two carbon atoms and then running a ring-opening reaction among these ethienyl groups, is repeated and (2) a method in which a step of substituting a triflate group for a hydrogen atom bound with a carbon atom at a specified position of a raw material, reacting the triflate group with furan or its derivative and in succession, oxidizing, is repeated. Examples of synthesizing the acene skeleton are shown below.

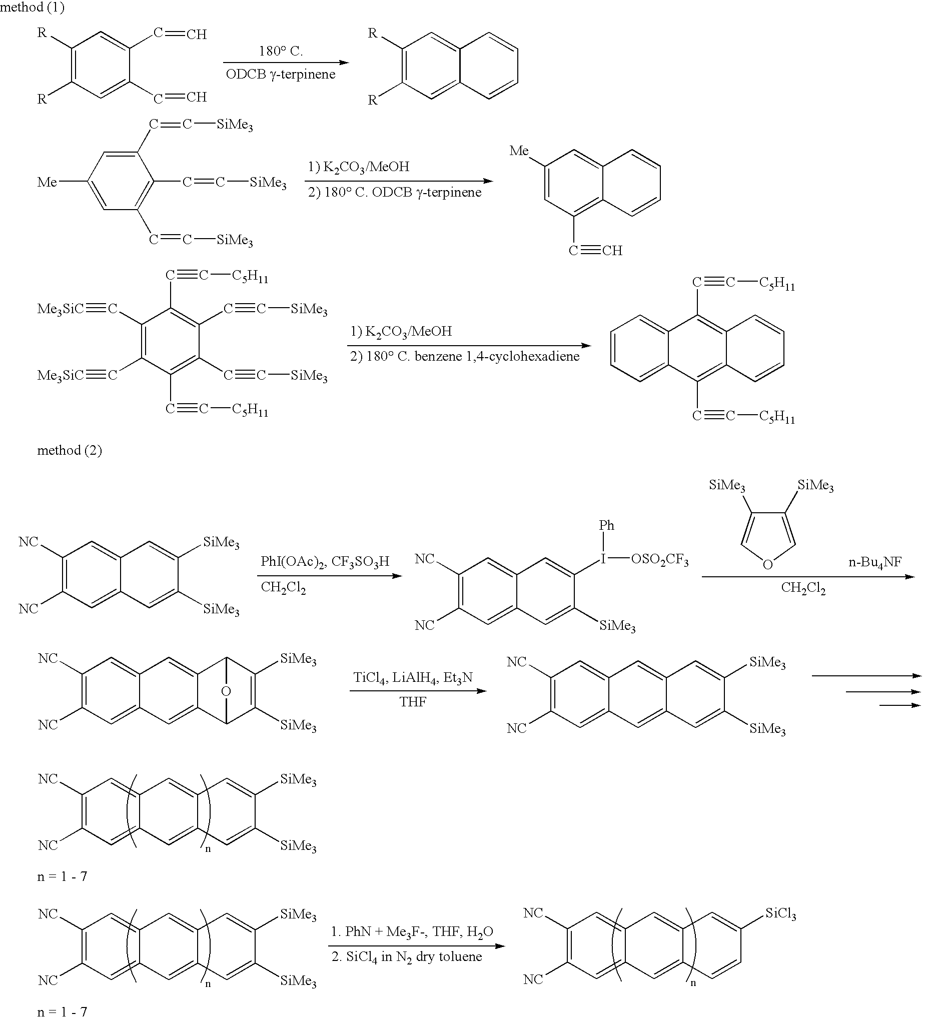

Also, because in the above method (2), a benzene ring of an acene skeleton is increased one by one, it is therefore possible to introduce side chains by using a raw material having these side chains and by increasing the number of condensed rings as described in the following synthetic example.

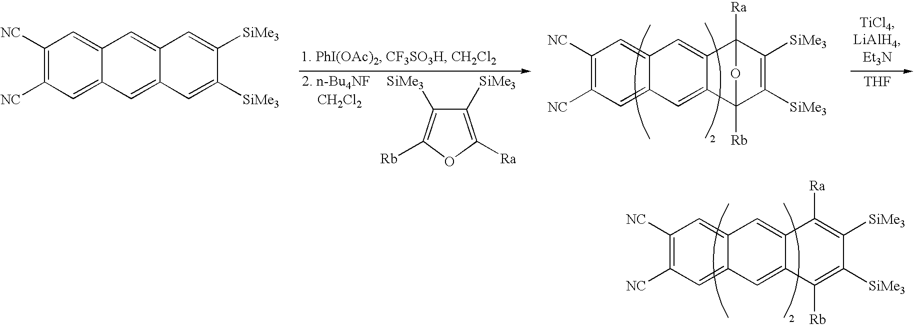

Rₐ and R_{b} are side chains.

Alternatively, in the reaction formula of the method (2), a starting compound having two acetonitrile groups and trimethylsilyl groups may be changed to a compound in which these groups are all a trimethylsilyl group. In addition, in the reaction formula, after a reaction using a furan derivative, by refluxing the reaction product under lithium iodide and DBU (1,8-diazabicyclo[5.4.0]undec-7-ene), a compound having one more benzene rings than the starting compound, and two substituted hydroxyl groups can be obtained. Further, when the hydroxyl group of this compound is brominated by the known method, and subjecting a bromo group to a Grignard reaction, a functional group can be introduced into a position of a bromo group.

In addition, for example, raw materials used above synthetic example are common reagents, which are commercially available from a reagent maker and can be utilized. For example, tetracene can be obtained at a purity of 97% or higher from Tokyo Kasei Kogyo Co., Ltd.

The organic silane compound obtained by such the method can be isolated and purified from the reaction solution by the known means, for example, elution, concentration, solvent extraction, fractionation, recrystallization, chromatography or the like.

For example, the organic thin film can be formed from the silane compound as follows.

First, the silica compound is dissolved in a non-aqueous method such as a hexane, chloroform, carbon tetrachloride and the like. In the resulting solution, the substrate on which the thin film is to be formed (preferably the substrate having active hydrogen of a hydroxyl group, a carboxyl group etc.) is immersed, and pulled out therefrom to form a coating film. Alternatively, the resulting solution may be coated on a substrate surface. Thereafter, this is washed with the non-aqueous solvent, washed with water, and dried by allowing to stand or heating, to fix the coating film as the organic thin film.

This organic thin film may be used directly as an electric material, or may be used by further subjecting to treatment such as electrolysis polymerization and the like. By using this silane compound, the organic thin film in which the network of Si—O—Si is formed, a distance between adjacent π-electron conjugate type molecules is small, and the compound is a highly ordered (crystallized), is obtained. In addition, when the units are linear, a distance between adjacent units can be further decreased, because adjacent units of the silane compounds are not bound. As a result, a more highly crystallized organic thin film can be obtained. Such organic thin film preferably is used in the organic thin film transistor.

In succession, the organic thin film transistor (organic TFT) of the present invention will be explained with reference to the drawings.

FIG. 2 is a conceptual view of one example of the organic TFT of the present invention. The organic TFT of FIG. 2 has a bottom gate and bottom contact type structure. In FIG. 2, 1 designates a substrate, 2 designates a gate electrode, 3 designates a gate insulating film, 4 designates an organic thin film and 5 and 6 designate source/drain electrodes. FIG. 2 is an example in which the bottom surface of the organic thin film is one surface on which source/drain electrodes are formed.

The structure of the organic TFT is not limited to the structure of FIG. 2. Examples of other structures include:

(1) a structure in which an organic thin film and source/drain electrodes are provided in this order on a substrate and a gate insulating film and a gate electrode are provided in this order on the organic thin film between the source/drain electrodes (an example in which the upper surface of the organic thin film is one surface on which source/drain electrodes are formed);

(2) a structure in which a gate electrode, a gate insulating film, an organic thin film and source/drain electrodes are provided in this order on a substrate (an example in which the bottom surface of the organic thin film is one surface and source/drain electrodes are formed on the other surface, i.e. the upper surface of the organic thin film); and

(3) a structure in which source/drain electrodes are provided on a substrate, an organic thin film and a gate insulating film are provided in this order so as to cover the source/drain electrodes and a gate electrode is provided on the gate insulating film (an example in which the upper surface of the organic thin film is one surface and source/drain electrodes are formed on the other surface, i.e. the bottom surface of the organic thin film).

The structural elements of the organic TFT of the present invention will be hereinafter explained in detail.

(Gate, Source/Drain Electrode)

No particular limitation is imposed on the gate, source/drain electrode materials and materials known in the fields concerned may be all used. Specific examples of these materials include metals such as gold, platinum, silver, copper and aluminum; high-melting point metals such as titanium, tantalum and tungsten; silicides and polycides of high-melting point metals; highly doped p-type or n-type silicon; conductive metal oxides such as ITO and NESA; and conductive polymers such as PEDOT.

There is no particular limitation to the film thickness and it may be properly adjusted to one (for example, 30 to 60 nm) that is usually used in the case of transistors.

The method of producing these electrodes may be properly selected corresponding to electrode materials. Examples of the method include vapor deposition, sputtering and coating.

(Gate Insulating Film)

There is no particular limitation to the gate insulating film and any film known in the fields concerned may be used. Examples of the gate insulating film include a silicon oxide film (thermally oxidized film, low temperature oxidized film: high temperature oxidized film such as LTO film etc.: HTO film), silicon nitride film, SOG film, PSG film, BSG film, BPSC film and the like; PZT, PLZT, ferroelectric or antiferromagnetic; low dielectrics such as SiOF-based film, SiOC-based film or CF-based film, HSQ (hydrogen silsesquioxane)-based film (inorganic type) formed by coating, MSQ (methyl silsesquioxane)-based film, PAE (polyarylene ether)-based film, BCB-based film, porous-film or CF-based film, or a porous film and the like.

There is no particular limitation to the film thickness and it may be properly adjusted to one (for example, 100 to 500 nm) that is usually used in the case of transistors.

The method of producing the gate insulating film may be properly selected corresponding to the type. Examples of the method include vapor deposition, sputtering and coating.

(Organic Thin Film)

As the material of the organic thin film, a side chain-containing type organic silane compound represented by the formula (I): R—SiX¹X²X³ is used, wherein RR represents aπ-electron conjugate type organic residue composed of 3 to 10 units whose units are a group derived from a monocyclic aromatic hydrocarbon, a group derived from a monocyclic heterocyclic compound or the combination thereof, the residue having at least one side chain and X¹, X² and X³, the same or different represent a group affording a hydroxyl group upon hydrolysis.

As the method of producing the organic thin film, usual methods such as a SAM method (for example, a LB method, vapor deposition, dipping, soaking, casting or CVD method) may be all applied. An appropriate method is set taking the costs of materials and mass-production into account.

The definition of each of these methods such as a SAM method, LB method, vapor deposition, dipping, soaking, casting and CVD method in this specification will be given below.

The SAM method is an abbreviation for Self-Assembled Monolayer and indicates a method forming a film by using a self-organizing material. This SAM method includes all of the LB method/dipping method/soaking method/casting method/QVD method.

The LB method is an abbreviation for Langmuir-Blodgett method and is a method in which an amphipathic material well balanced between hydrophobic groups and hydrophilic groups is developed on the surface of the water to produce a film of a molecular single layer which is the so-called monomolecular layer, and then, the film is transferred to a substrate.

The vapor deposition method is a method in which a raw material is heated to form its vapor, which is then deposited on a desired region. In the case of using an organic semiconductor material, a vapor deposition method using resistance heating may be used.

The soaking method means a method in which a substrate is simply soaked in a solution and then taken out of the solution to form a film.

The casting method means a method in which a solution containing a raw material is dripped on a desired region and is then dried to form a film. This method includes an ink jetting method.

The CVD method means a method in which a solution is heated/vaporized in a closed container or a closed space and the gasified molecules are adsorbed in a vapor phase to the surface of a substrate.

Also, examples of the method of producing the organic TFT include a method involving:

(1) a step of forming a gate electrode on a substrate, a step of a gate insulating film on the gate electrode, a step of forming an organic thin film on the gate insulating film and a step of forming source/drain electrodes before or after the organic thin film is formed; or

(2) a step of forming source/drain electrodes on a substrate, a step of forming an organic thin film before or after the source/drain electrodes are formed, a step of a gate insulating film on the organic thin film and a step of forming a gate electrode on the gate insulating film.

The silane compound and the method of producing the silane compound will be explained by way of examples. The methods of synthesizing silane compounds containing a phenyl group or a thiophene group in Examples 1 to 4 and silane compounds containing a residue derived from naphthacene or pentacene in Examples 5 to 7 according to the present invention will be described. The silane compound of the present invention is not limited to the compounds of the following examples.

### EXAMPLES

### Example 1 Synthesis of an Organic Silane Compound Represented by the Formula (a)

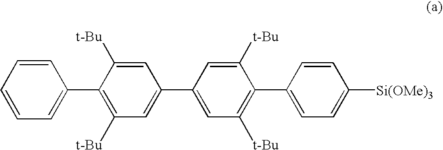

The inscribed compound was synthesized by the following method. First, 1 M of 2-bromo-2-methyl-propane was dissolved in carbon tetrachloride, to which 1 M of metal magnesium was added, and the mixture was reacted at 60° C. for one hour to form a Grignard's reagent.

In succession, 0.5 M of m-dichlorobenzene was added to the mixture, which was then reacted at 20° C. for one hour to form m-di(tert-butyl)-benzene.

In succession, 1 M of NBS and 1 M of AIBN were added to a carbon tetrachloride solution containing 0.5 M of the above m-di(tert-butyl)-benzene to synthesize 2,5-dibromo-1,3-di-tert-butyl-benzene.

Then, metal magnesium was added to a carbon tetrachloride solution containing 0.2 M of the above 2,5-dibromo-1,3-di-tert-butyl-benzene and the mixture was reacted at 60° C. for one hour. Then, 0.2 M of 2,5-dibromo-1,3-di-tert-butyl-benzene was added to the mixture, which was then reacted at 25° C. for one hour to synthesize 3,5,3′,5′-tetra-tert-butyl-biphenyl.

0.25 M of NBS and 0.25 M of AIBN were added in a carbon tetrachloride solution containing 0.1 M of the above 3,5,3′,5′-tetra-tert-butyl-biphenyl and the mixture was reacted at 60° C. for 6 hours to form 3,5,3′,5′-tetra-tert-butyl-4,4′-dibromo-biphenyl.

Then, 0.1 M of 3,5,3′,5′-tetra-tert-butyl-4,4′-dibromo-biphenyl was added in a THF solution containing 0.1 M of a Grignard's reagent synthesized from metal magnesium and bromobenzene and the mixture was reacted at 40° C. for 2 hours to synthesize 2′,6′,2″,6″-tetra-tert-butyl-[1,1′; 4,4″; 1″,1″′]quaterphenyl.

Then, 0.1 M of NBS and 0.1 M of AIBN were added in a carbon tetrachloride solution containing 0.1 M of the above 2′,6′,2″,6″-tetra-tert-butyl-[1,1′; 4,4″;1″,1′″]quaterphenyl and the mixture was reacted at 60° C. for 1.5 hours. Then, metal magnesium was added to the reaction mixture to form a Grignard's reagent. This reagent was added to a THF solution containing 0.1 M of chlorotrimethoxysilane and the mixture was reacted at 45° C. for 2 hours to synthesize the inscribed compound.

The scheme of the above synthetic method is described below.

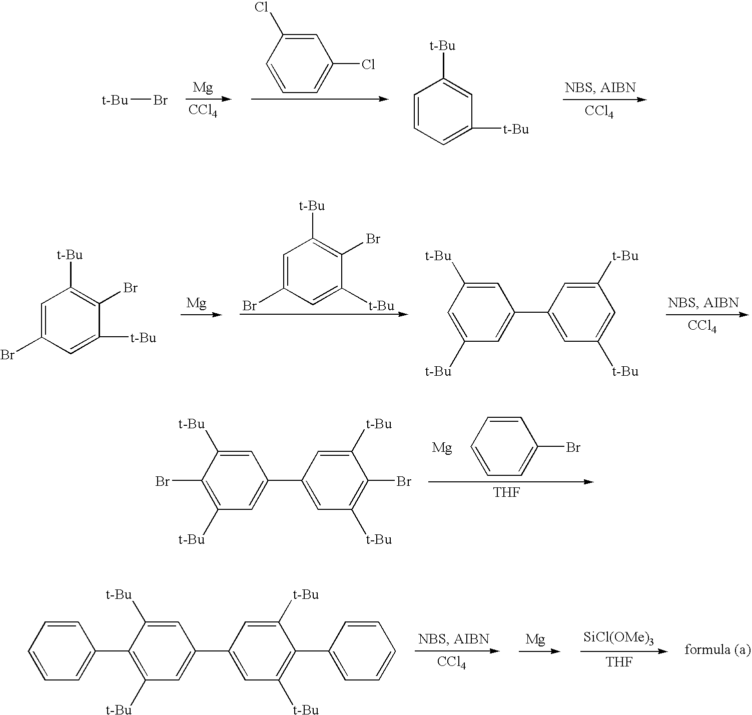

In the above scheme, t-Bu and Me mean tert-butyl and methyl respectively.

The infrared absorption spectrum of the resulting compound was measured, to find that an absorption derived from SiC was observed at 1090 cm⁻¹, thereby confirming that the compound had a SIC bond.

The nuclear magnetic resonance (NMR) of the resulting compound was measured.

7.5 ppm (m) (4H aromatic)

7.3 ppm (m) (8H aromatic)

7.2 ppm (m) (1H aromatic)

3.6 ppm (m) (9H ethoxy group methylene group)

1.5 ppm (m) (36H ethoxy group methyl group)

Moreover, the molecular occupying volumes of the main skeleton and side chain of the resulting compound, and the ratio (volume ratio) of the molecular occupying volume of the side chain to that of the main skeleton are shown in Table 2. Each molecular occupying volume of the side chain and main skeleton was calculated as follows.

As to the main skeleton, first, the whole of the main skeleton was approximated to a cylinder to suppose that the volume of the cylinder as the molecular occupying volume of the main skeleton. Specifically, the volume of the cylinder obtained by rotating 360° on the major axis (an axis which is perpendicular to the axis formed by π electrons and passes between two atoms (excluding hydrogen) having the longest molecular length) of the structural formula of the molecule constituting the main skeleton was defined as the molecular occupying volume of the main skeleton.

As to the side chain, the whole of the side chain was approximated to a cone to suppose that the volume of the cone as the molecular occupying volume of the side chain. Specifically, the volume of the cone obtained by rotating by 360° on, as the center axis, a straight line across two points, namely, the atom of the main skeleton bound with the side chain and the atom directly bound with the main skeleton was defined as the molecular occupying volume of the side chain.

In this case, the interatomic distance was calculated after the structures of the cylinder and cone were optimized by molecular orbital calculation (AM1).

### Example 0.2 Synthesis of an Organic Silane Compound Represented by the formula (b)

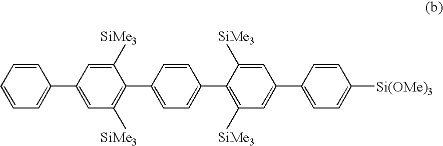

The inscribed compound was synthesized by the following method. First, 0.5 M of m-dichlorobenzene was dissolved in carbon tetrachloride, to which 0.5 M of metal magnesium was added, and the mixture was reacted at 60° C. for one hour to form a Grignard's reagent.

In succession, 1 M of chlorotrimethylsilane was added to the mixture, which was then reacted at 20° C. for one hour to form m-di(trimethylsilyl)-benzene.

In succession, 1 M of NBS and 1 M of AIBN were added to a carbon tetrachloride solution containing 0.5 M of the above m-di(trimethylsilyl)-benzene to synthesize 5-bromo-1,3-di-trimethylsilyl-benzene.

Then, 0.2 M of metal magnesium was added to a carbon tetrachloride solution containing 0.2 M of the above 5-bromo-1,3-di-trimethylsilyl-benzene and the mixture was reacted at 60° C. for one hour. Then, 0.2 M of bromobenzene was added to the mixture, which was then reacted at 25° C. for one hour to synthesize 3,5-bis-trimethylsilyl-biphenyl.

1 M of NBS and 1 M of AIBN were added in a carbon tetrachloride solution containing 0.5 M of the above 3,5-bis-trimethylsilyl-biphenyl and the mixture was reacted at 50° C. for one hour to synthesize 4-bromo-3,5-bis-trimethylsilyl-biphenyl.

Then, a THF solution containing 0.2 M of the above 4-bromo-3,5-bis-trimethylsilyl-biphenyl was added to a THF solution containing 0.1 M of a Grignard's reagent formed from 0.5 M of p-dibromobenzene and 0.5 M of metal magnesium and the mixture was reacted at 25° C. for one hour to synthesize 2′,6′,2″′,6″′-tetrakis-trimethylsilyl-[1,4′;1′,1″;4″,1″′;4″′,1″″]quinquephenyl.

Then, 0.1 M of NBS and 0.1 M of AIBN were added in a carbon tetrachloride solution containing 0.1 M of the above 2′,6′,2″′,6″′-tetrakis-trimethylsilyl-[1,4′;1′,1″;4″,1″′;4″′,1″″]quinquephenyl and the mixture was reacted at 60° C. for 1.5 hours. Then, metal magnesium was added to the reaction mixture to form a Grignard's reagent. This reagent was added to a THF solution containing 0.1 M of chlorotrimethoxysilane and the mixture was reacted at 45° C. for 2 hours to synthesize the inscribed compound.

The scheme of the above synthetic method is described below.

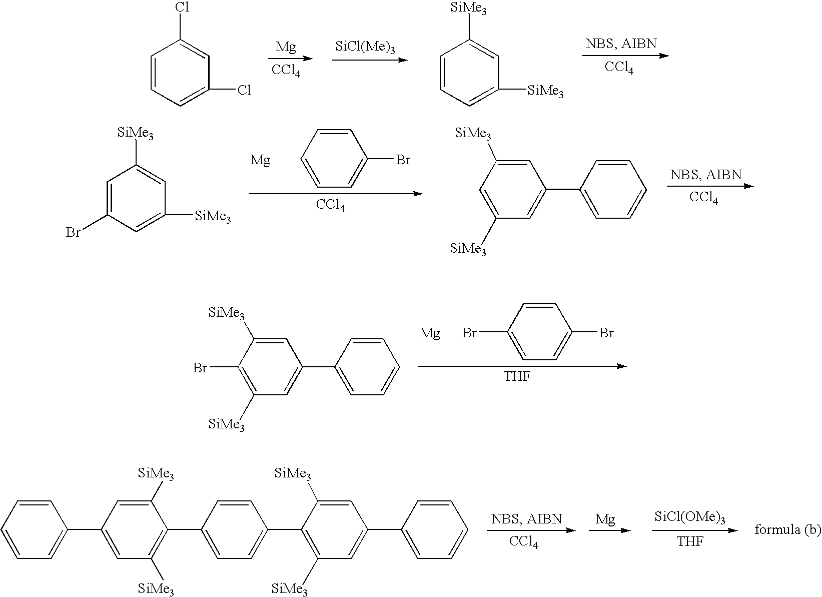

The nuclear magnetic resonance (NMR) of the resulting compound was measured.

7.5 ppm (m) (4H aromatic)

7.5 ppm (m) (8H aromatic)

7.3 ppm (m) (4H aromatic)

7.2 ppm (m) (1H aromatic)

3.6 ppm (m) (9H ethoxy group methylene group)

1.1 ppm (m) (36H methyl group)

From the above results, it was confirmed that the obtained compound was a compound represented by the formula (b).

Moreover, the molecular occupying volumes of the main skeleton and side chain of the resulting compound, and the ratio (volume ratio) of the molecular occupying volume of the side chain to that of the main skeleton are shown in Table 2.

### Example 3 Synthesis Of an Organic Silane Compound Represented by the Formula (c)

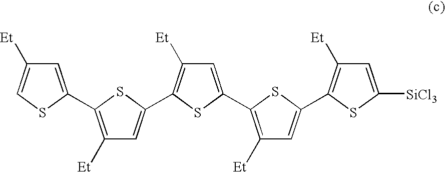

The inscribed compound was synthesized by the following method. First, a Grignard's reagent was synthesized from 3-bromothiophene and metal magnesium and then chloroethane were added to the reagent to synthesize 3-ethylthiophene.

In succession, 1 M of NBS and 1 M of AIBN were added to a carbon tetrachloride solution containing 0.5 M of the above 3-ethylthiophene. The mixture was reacted at 55° C. for 2 hours to synthesize 2-bromo-3-ethylthiophene and also reacted at 55° C. for 6 hours to synthesize 2,5-dibromo-3-ethylthiophene separately.

A Grignard's reagent was formed from the above 2-bromo-3-ethylthiophene and metal magnesium. 0.4 M of the above Grignard's reagent was added in a THF solution containing 0.2 M of the above 2,5-dibromo-3-ethylthiophene and the mixture was reacted at 30° C. for 4 hours to synthesize 3,4′,4″-triethyl-[2,2′;5′2″]terthiophene.

Moreover, 0.1 M of the above 3,4′,4″-triethyl-[2,2′;5′2″]terthiophene was dissolved in carbon tetrachloride and 0.3 M of NBS and 0.3 M of AIBN were added to the mixture, which was then reacted at 55° C. for 2 hours to synthesize 3,4′,4″-triethyl-5,5″-dibromo-[2,2′;5′2″]terthiophene. 0.1 M of the above 3,4′,4″-triethyl-5,5″-dibromo-[2,2′;5′2″]terthiophene was added in a THF solution containing 0.1 M of the above Grignard's reagent formed from the above 2-bromo-3-ethylthiophene and metal magnesium and the mixture was reacted at 60° C. for 5 hours to synthesize 3,4′,4″,4″′,4″″-pentaethyl-[2,2′;5′,2″;5″,2″′;5″′,2″″]quinquethiophene.

Then, 200 mM of NBS and 200 mM of AIBN were added in a carbon tetrachloride solution containing 50 mM of 3,4′,4″,4″′,4″″-pentaethyl-[2,2′;5′,2″;5″,2″′;5″′,2″″]quinquethiophene and the mixture was reacted at 60° C. for one hour. Then, metal magnesium was added to the reaction mixture to form a Grignard's reagent. 50 mM of this reagent was added to a THF solution containing 50 mM of tetrachlorosilane and the mixture was reacted at 50° C. for 2 hours to synthesize the inscribed compound.

The scheme of the above synthetic method is described below.

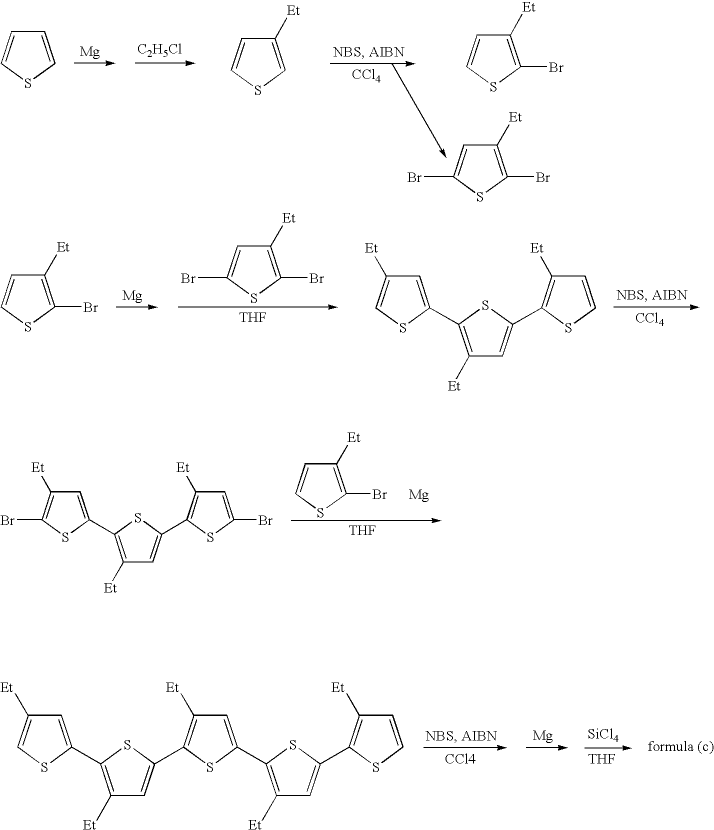

In the above scheme, Et means ethyl.

The infrared absorption spectrum of the resulting compound was measured, to find that an absorption derived from SiC was observed at 1090 cm⁻¹, thereby confirming that the compound had a SIC bond.

Moreover, the nuclear magnetic resonance (NMR) of the resulting compound was measured. It was impossible to measure the NMR of the obtained compound directly because the compound had high reactivity. Therefore, the NMR was measured after it was reacted with ethanol (the generation of hydrogen chloride was confirmed) to convert the terminal chlorine into an ethoxy group.

6.7 ppm (m) (6H aromatic)

3.6 ppm (m) (6H ethoxy group methylene group)

2.6 ppm (m) (10H ethyl group methylene group)

1.4 ppm (m) (36H ethoxy group methyl group)

1.2 ppm (m) (15H ethyl group, terminal methyl group)

From these results, it was confirmed that the obtained compound was a compound represented by the formula (c).

Moreover, the molecular occupying volumes of the main skeleton and side chain of the resulting compound, and the ratio (volume ratio) of the molecular occupying volume of the side chain to that of the main skeleton are shown in Table 2.

### Example 4 Synthesis of an Organic Silane Compound Represented by the Formula (d)

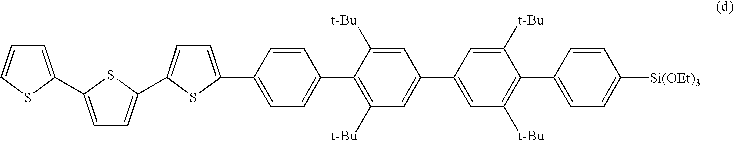

The inscribed compound was synthesized by the following method. First, 50 mM of NBS and 50 mM of AIBN were added to a carbon tetrachloride solution containing 20 mM of 2′,6′,2″,6″-tetra-tert-butyl-[1,1′;4,4″;1″,1″′]quaterphenyl which was the intermediate in Example 1 and the mixture was reacted at 60° C. for 1.5 hours. Metal magnesium was added to the reaction mixture to form a Grignard's reagent, to which 20 mM of 2-bromoterthiophene was added and the mixture was reacted at 45° C. for 2 hours to synthesize 5-(2′,6′,2″,6″-tetra-tert-butyl-[1,1′;4′,4″;1″,1″′]quaterphenyl-4-yl)-[2,2′;5′,2″]terthiophene.

In succession, 20 mM of NBS and AIBN were added in a carbon tetrachloride solution containing 10 mM of the above 5-(2′,6′,2″,6″-tetra-tert-butyl-[1,1′;4′,4″;1″,1″′]quaterphenyl-4-yl)-[2,2′;5′,2″]terthiophene and the mixture was reacted at 50° C. for 1.5 hours. Then, metal magnesium was added to the reaction mixture to form a Grignard's reagent, which was then added in a THF solution containing 10 mM of chlorotrimethoxysilane and the mixture was reacted at 45° C. for 2 hours to synthesize the inscribed compound.

The scheme of the above synthetic method is described below.

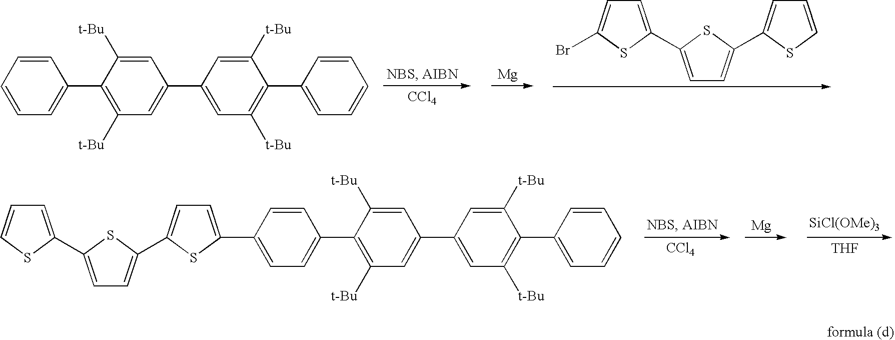

Moreover, the nuclear magnetic resonance (NMR) of the resulting compound was measured.

7.5 ppm (m) (6H aromatic phenyl group)

7.4 ppm (m) (6H aromatic thiophene group)

7.3 ppm (m) (6H aromatic phenyl group)

7.2 ppm (m) (1H aromatic thiophene group)

3.6 ppm (m) (6H ethoxy group methylene group)

1.5 ppm (m) (9H ethoxy group methyl group)

1.2 ppm (m) (36H methyl group)

From these results, it was confirmed that the obtained compound was a compound represented by the formula (d).

Moreover, the molecular occupying volumes of the main skeleton and side chain of the resulting compound, and the ratio (volume ratio) of the molecular occupying volume of the side chain to that of the main skeleton are shown in Table 2.

### Example 5 Synthesis of an Organic Silane Compound Represented by the Formula (e)

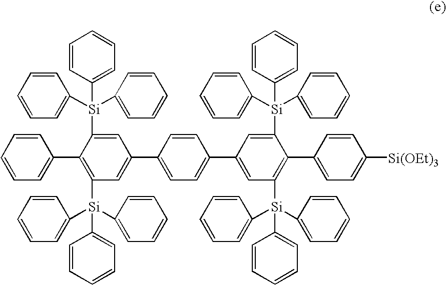

The inscribed compound was synthesized by the following method. First, 0.5 M of m-dichlorobenzene was dissolved in carbon tetrachloride, to which 0.5 M of metal magnesium was added and the mixture was reacted at 60° C. for one hour to form a Grignard's reagent.

In succession, 1 M of chlorotriphenylsilane was added to the reaction mixture, which was then reacted at 20° C. for 2 hours to form m-di(triphenylsilyl)-benzene.

In succession, 1 M of NBS and 1 M of AIBN were added in a carbon tetrachloride solution containing 0.5 M of the above m-di(triphenylsilyl)-benzene to synthesize 5-bromo-1,3-di-triphenylsilylbenzene.

0.3 M of metal magnesium was added in a carbon tetrachloride solution containing 0.3 M of the above 5-bromo-1,3-di-triphenylsilylbenzene and the mixture was reacted at 60° C. for one hour. Then, 0.3 M of bromobenzene was added to the mixture, which was then reacted at 25° C. for 2 hours to synthesize 3,5-bis-triphenylsilylbiphenyl.

Moreover, 1 M of NBS and 1 M of AIBN were added in a carbon tetrachloride solution containing 0.5 M of the above 3,5-bis-triphenylsilylbiphenyl and the mixture was reacted at 60° C. for 2 hours to synthesize 4-bromo-3,5-bis-triphenylsilylbiphenyl.

Then, a THF solution containing 0.3 M of the above 4-bromo-3,5-bis-triphenylsilylbiphenyl was added in a THF solution containing 0.2 M of a Grignard's reagent formed from 0.5 M of p-dibromobenzene and 0.5 M of metal magnesium and the mixture was reacted at 25° C. for one hour to synthesize 2′,6′,2″′,6″′-tetrakis-triphenylsilyl[1,4′;1′,1″;4″,1″′;4″′,1″″]quinquephenyl.

Moreover, 0.1 M of NBS and 0.1 M of AIBN were added to a carbon tetrachloride solution containing 0.1 M of the above 2′,6′,2″′,6″′-tetrakis-triphenylsilyl[1,4′;1′,1″;4″,1″′;4″′,1″″]quinquephenyl and the mixture was reacted at 60° C. for 3 hours. Metal magnesium was added to the reaction mixture to form a Grignard's reagent, which was then added in a THF solution containing 0.1 M of chlorotrimethoxysilane and the mixture was reacted at 45° C. for 2 hours to synthesize the inscribed compound.

Moreover, the nuclear magnetic resonance (NMR) of the resulting compound was measured.

7.9 ppm (m) (4H quinquephenyl group)

7.6 ppm (m) (28H quinquephenyl group and triarylsilyl group)

7.5 ppm (m) (4H quinquephenyl group)

7.4 ppm (m) (36H quinquephenyl group and triarylsilyl group)

7.3 ppm (m) (4H quinquephenyl group)

3.6 ppm (m) (9H ethoxy group methylene group)

1.1 ppm (m) (36H methyl group)

From these results, it was confirmed that the obtained compound was a compound represented by the formula (e).

Moreover, the molecular occupying volumes of the main skeleton and side chain of the resulting compound, and the ratio (volume ratio) of the molecular occupying volume of the side chain to that of the main skeleton are shown in Table 2.

### Example 6 Synthesis of an Organic Silane Compound Represented by the Formula (f)

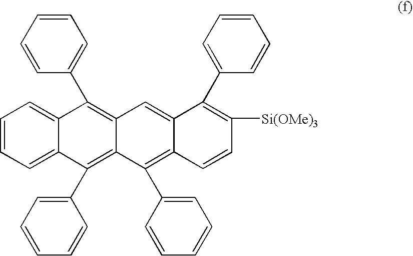

The inscribed compound was synthesized by the following method.

First, 20 mM of NBS and 20 mM of AIBN were added in a carbon tetrachloride solution containing 5 mM of 5,6,11,12-tetraphenyl-naphthacene and the mixture was reacted at 65° C. for one hour to synthesize 3-bromo-5,6,11,12-tetraphenyl-naphthacene. In succession, 2 mM of metal magnesium was added in a dichloroethane solution containing 2 mM of the above 3-bromo-5,6,11,12-tetraphenyl-naphthacene to form a Grignard's reagent. Then, 2 mM of chlorotrimethoxysilane was added to the Grignard's reagent and the mixture was reacted at 20° C. for 2 hours to synthesize the inscribed compound.

The scheme of the above synthetic method is described below.

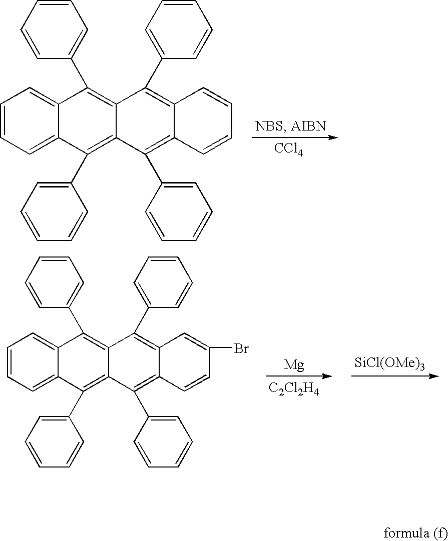

In the above scheme, Me means methyl.

The infrared absorption spectrum of the resulting compound was measured, to find that an absorption derived from SiC was observed at 1090 cm⁻¹, thereby confirming that the compound had a SiC bond.

Moreover, the nuclear magnetic resonance (NMR) of the resulting compound was measured.

7.9 ppm (m) (4H aromatic)

7.5 ppm (m) (8H aromatic)

7.4 ppm (m) (3H aromatic)

7.3 ppm (m) (8H aromatic)

7.2 ppm (m) (4H aromatic)

3.6 ppm (m) (9H methoxy group methyl group)

From these results, it was confirmed that the obtained compound was a compound represented by the formula (f).

Moreover, the molecular occupying volumes of the main skeleton and side chain of the resulting compound, and the ratio (volume ratio) of the molecular occupying volume of the side chain to that of the main skeleton are shown in Table 2.

### Example 7 Synthesis of an Organic Silane Compound Represented by the Formula (g)

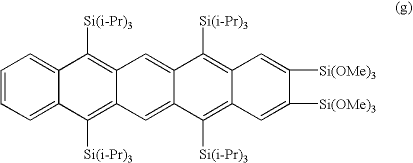

The inscribed compound was synthesized by the following method.

### Reference Example

First,

2,3,6,7-tetra(trimethylsilyl)-5,8-di(triisopropylsilyl) naphthalene was synthesized by the following method.

To mention in detail, first, a 200 ml glass flask equipped with a stirrer, a reflux condenser, a temperature gauge and a dropping funnel was charged with 0.4 M of magnesium, 100 mL of HMPT (hexamethylphosphorous acid triamide), 20 mL of THF and I₂ (catalyst), and 0.1 M of 1,2,4,5-tetrachlorobenzene. 0.4 M of chlorotrimethylsilane was added dropwise to the mixture at 80° C., which was then stirred for 30 minutes and refluxed at 130° C. for 4 days to thereby synthesize 1,2,4,5-tetra(trimethylsilyl)benzene.

In succession, a 200 mL eggplant-shape flask was charged with 20 mM of i-PrNH₂, 50 mM of PhI(OAc)₂(diacetoxyiodobenzene) and 50 mL of dichloromethane and then, 50 mM of CF₃CO₂H(T_{f}OH) was added dropwise to the mixture at 0° C., followed by stirring for 2 hours.

Then, 10 mL of a dichloromethane solution containing 50 mM of the above 1,2,4,5-tetra(trimethylsilyl)benzene was added dropwise to the mixture at 0° C. and the resulting mixture was stirred at ambient temperature for 2 hours to synthesize phenyl[2,4,5-tris(trimethylsilyl)phenyl]iodonium trifluorate.

In succession, a 50 mL eggplant-shape flask was charged with a THF solution containing 2.0 M of Bu₄NF and 10 mL of a dichloromethane solution containing 5 mM of the above phenyl[2,4,5-tris(trimethylsilyl)phenyl]iodonium trifluorate and 10 mM of 2,5-tri(isopropyl)silyl-3,4-di(trimethylsilyl)furan was added dropwise to the THF solution at 0° C. and the mixture was stirred for 30 minutes to allow the reaction to proceed. After the reaction was finished, the reaction solution was extracted with dichloromethane and water and the extract was refined by column chromatography to synthesize a 1,4-dihydro-1,4-epoxynaphthalene derivative.

Then, a 50 mL glass flask equipped with a stirrer, a reflux condenser, a temperature gauge and a dropping funnel was charged with 10 mL of a THF solution containing 1 mM of lithium iodide and 10 mM of DBU and 1 mM of the above 1,4-dihydro-1,4-epoxynaphthalene derivative was added to the mixture, which was then refluxed in a nitrogen atmosphere for 3 hours to progress the reaction. After the reaction was finished, the reaction mixture was extracted and water was removed using MgSO₄ to synthesize the target 2,3,6,7-tetra(trimethylsilyl)-5,8-di(triisopropylsilyl)naphthalene.

### Synthetic Examples of the Compound Represented by the Formula (g)

Next, the same synthetic method as in the case of synthesizing 2,3,6,7-tetra(trimethylsilyl)-5,8-di(triisopropylsilyl)naphthalene from 1,2,4,5-tetra(trimethylsilyl)benzene in Reference Example was used except that the above 2,3,6,7-tetra(trimethylsilyl)-5,8-di(triisopropylsilyl) was used as starting material and 3,4-di(trimethylsilyl)furan was used in place of 2,5-tri(isopropyl)silyl-3,4-di(trimethylsilyl)furan, to thereby synthesize 2,3,7,8-tetra(trimethylsilyl)-6,9-di(triisopropylsilyl) anthracene.

Moreover, the same method as in the case of synthesizing 2,3,7,8-tetra(trimethylsilyl)-6,9-di(triisopropylsilyl) anthracene from 2,3,6,7-tetra(trimethylsilyl)-5,8-di(triisopropylsilyl) naphthalene was applied except that 2,5-tri(isopropyl) silyl-3,4-di(trimethylsilyl)furan was used in place of 3,4-di(trimethylsilyl)furan, to thereby synthesize 2,3,8,9-tetra(trimethylsilyl)-5,7,10,12-di(triisopropylsilyl)tetracene.

Moreover, the same method as in the case of synthesizing 2,3,8,9-tetra(trimethylsilyl)-5,7,10,12-tert(triisopropylsilyl)tetracene from 2,3,7,8-tetra(trimethylsilyl)-6,9-di(triisopropylsilyl)anthracene was applied except that 3,4-di(trimethylsilyl)furan was used in place of 2,5-tri(isopropyl) silyl-3,4-di(trimethylsilyl)furan, to thereby synthesize 2,3,9,10-tetra(trimethylsilyl)-5,7,12,14-tetra(triisopropylsilyl)pentacene.

Then, 10 mM of the above 2,3,9,10-tetra(trimethylsilyl)-5,7,12,14-tetra(triisopropylsilyl)pentacene was dissolved in a THF solvent containing a small amount of water and PhNMe₃F and the mixture was stirred to synthesize 5,7,12,14-tetra(triisopropylsilyl)pentacene.

Moreover, a 200 ml eggplant-shape flask was charged with 5 ml of dried THF, 5 mM of the above 5,7,12,14-tetra(triisopropylsilyl)pentacene and magnesium and the mixture was stirred for one hour to form a Grignard's reagent in a nitrogen atmosphere. Then, a 100 ml eggplant-shape flask equipped with a stirrer, a reflux condenser, a temperature gauge and a dropping funnel was charged with 5 mM of chlorotrimethoxysilane and 30 ml of THF and the mixture was ice-cooled. Then, the above Grignard's reagent was added to the mixture, which was then aged at 30° C. for one hour. Then, the reaction solution was filtered under reduced pressure to remove magnesium chloride from the reaction solution and THF and unreacted chlorotrimethoxysilane were removed from the solution by stripping to obtain the target compound at a yield of 10%.

The scheme of the above synthetic method is described below.

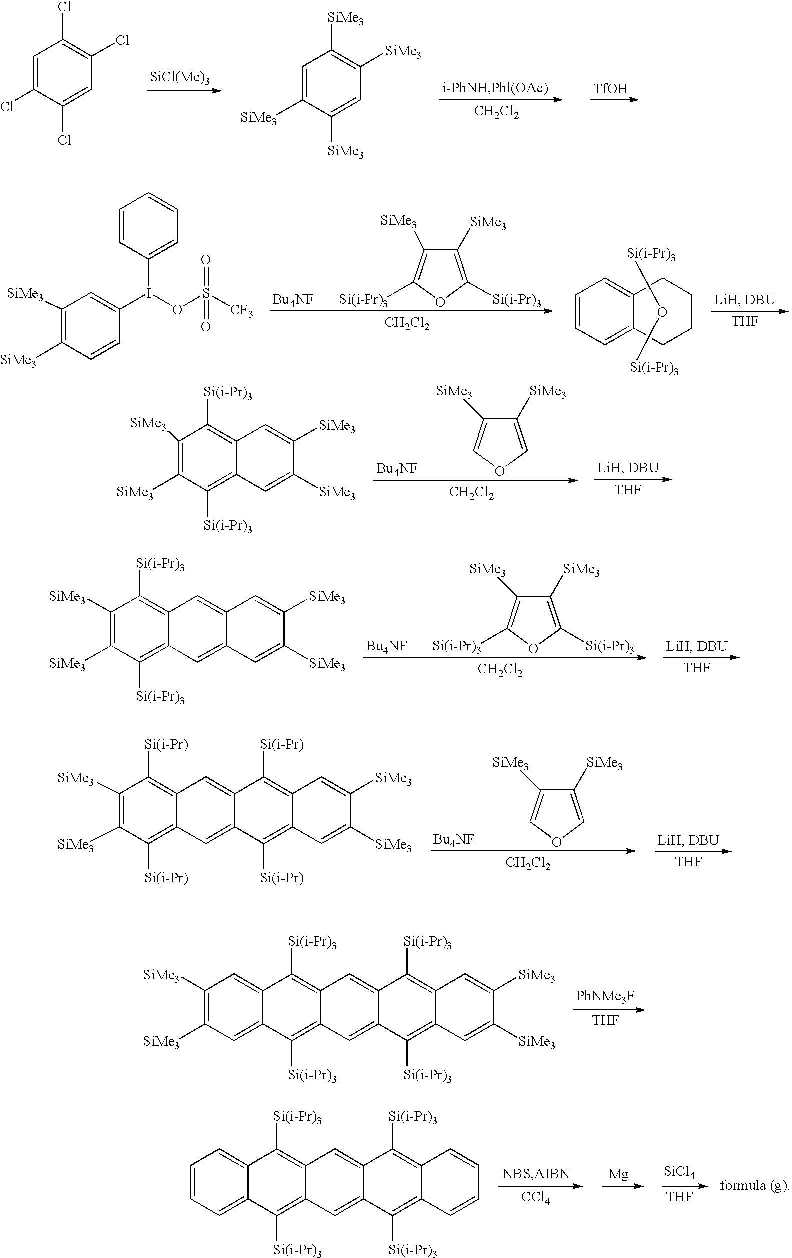

In the above scheme, Me means methyl, i-Pr means isopropyl, Ph means phenyl, Ac means acetyl and Bu means butyl.

The infrared absorption spectrum of the resulting compound was measured, to find that an absorption derived from SiC was observed at 1090 cm⁻¹, thereby confirming that the compound had a SiC bond.

Moreover, the nuclear magnetic resonance (NMR) of the resulting compound was measured.

7.9 ppm (m) (6H aromatic)

7.4 ppm (m) (2H aromatic)

3.6 ppm (m) (9H methoxy group methyl group)

1.5 ppm (m) (48H isopropyl group)

1.2 ppm (m) (12H isopropyl group)

From these results, it was confirmed that the obtained compound was a compound represented by the formula (g).

Moreover, the molecular occupying volumes of the main skeleton and side chain of the resulting compound, and the ratio. (volume ratio) of the molecular occupying volume of the side chain to that of the main skeleton are shown in Table 2.

### Example 8 Synthesis of an Organic Silane Compound Represented by the Formula (h)

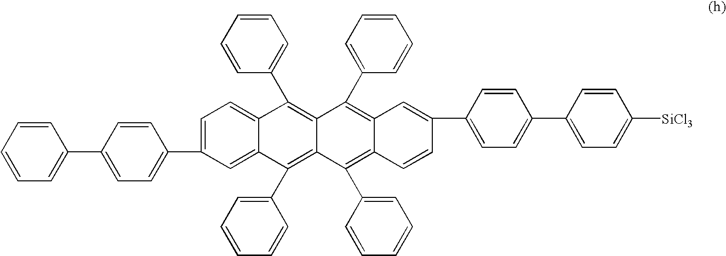

The inscribed compound was synthesized by the following method.

40 mM of NBS and 40 mM of AIBN were added in a carbon tetrachloride solution containing 5 mM of 5,6,11,12-tetraphenyl-naphthacene and the mixture was reacted at 65° C. for 6 hours to synthesize 3,8-dibromo-5,6,11,12-tetraphenyl-naphthacene.

In succession, metal magnesium was added in a carbon tetrachloride solution containing 10 mM of bromodiphenyl and the mixture was reacted at 60° C. for one hour to form a Grignard's reagent. 4 mM of the above 3,8-dibromo-5,6,11,12-tetraphenyl-naphthacene was added to the mixture, which was reacted at 20° C. for 8 hours to synthesize 2,8-bis-biphenyl-4-yl-5,6,11,12-tetraphenyl-naphthacene.

In succession, 10 mM of NBS and 10 mM of AIBN were added in a carbon tetrachloride solution containing 2 mM of the above 2,8-bis-biphenyl-4-yl-5,6,11,12-tetraphenyl-naphthacene and the mixture was reacted at 65° C. for one hour and then, metal magnesium was used to form a Grignard's reagent. Then, the Grignard's reagent was added in 2 mM of tetrachlorosilane and the mixture was reacted at 45° C. for 2 hours to synthesize the inscribed compound.

The scheme of the above synthetic method is described below.

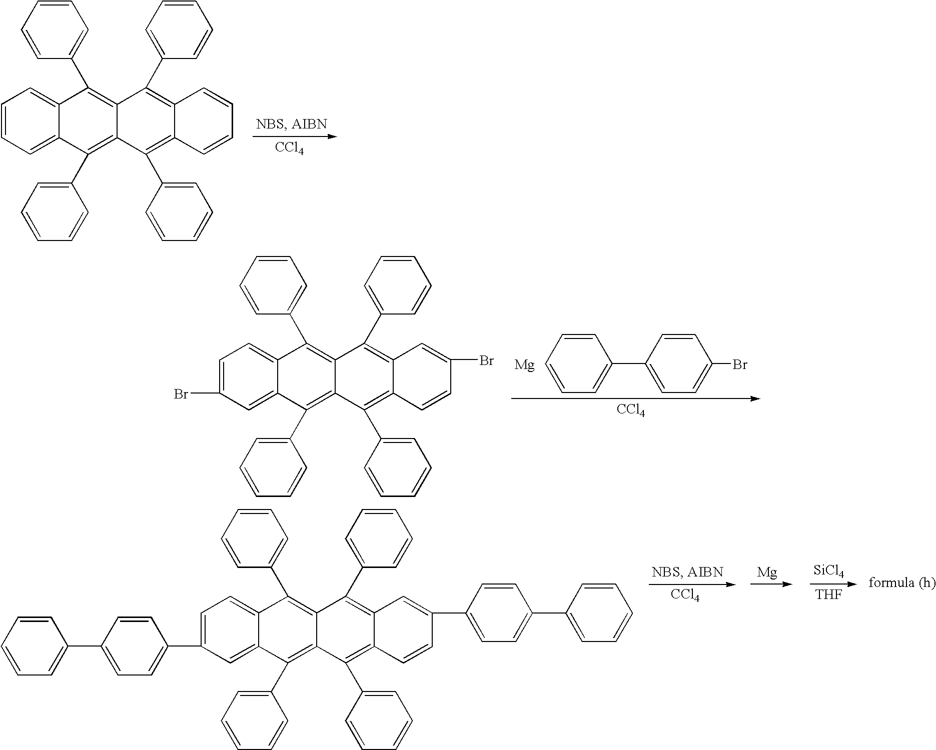

The infrared absorption spectrum of the resulting compound was measured, to find that an absorption derived from SiC was observed at 1075 cm⁻¹, thereby confirming that the compound had a SiC bond.

Moreover, the nuclear magnetic resonance (NMR) of the resulting compound was measured. It was impossible to measure the NMR of the obtained compound directly because the compound had high reactivity. Therefore, the NMR was measured after it was reacted with ethanol (the generation of hydrogen chloride was confirmed) to convert the terminal chlorine into an ethoxy group.

8.1 ppm (m) (2H aromatic tetracene skeleton)

7.9 ppm (m) (2H aromatic tetracene skeleton)

7.6 ppm (m) (2H aromatic tetracene skeleton)

7.5 ppm (m) (8H aromatic phenyl group)

7.4 ppm (m) (12H aromatic phenyl group)

7.3 ppm (m) (12H aromatic phenyl group)

7.2 ppm (m) (5H aromatic phenyl group)

3.6 ppm (m) (6H ethoxy group methylene group)

1.4 ppm (m) (9H ethoxy group methyl group)

From these results, it was confirmed that the obtained compound was a compound represented by the formula (h).

Moreover, the molecular occupying volumes of the main skeleton and side chain of the resulting compound, and the ratio (volume ratio) of the molecular occupying volume of the side chain to that of the main skeleton are shown in Table 2.

### Example 9 Synthesis of an Organic Silane Compound Represented by the Formula (i)

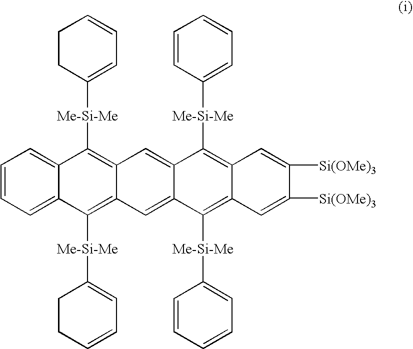

The inscribed compound was synthesized by the following method. First, 2,3,6,7-tetra(trimethylsilyl)-5,8-di(dimethylphenylsilyl)naphthalene was synthesized in the same method as in Reference Example of Example 7 except that Ph-Si(CH₃)₂NH was used in place of i-PrNH. The inscribed compound was obtained by applying the same method as in the synthetic example of Example 7 except that the above 2,3,6,7-tetra(trimethylsilyl)-5,8-di(dimethylphenylsilyl)naphthalene was used as the starting material and the sample obtained on the way of synthesis in place of 2,3,6,7-tetra(trimethylsilyl)-5,8-di(triisopropylsilyl)naphthalene.

The nuclear magnetic resonance (NMR) of the resulting compound was measured.

7.9 ppm (m) (6H pentacene)

7.5 ppm (m) (8H dimethylphenylsilyl group)

7.4 ppm (m) (14H pentacene and dimethylphenylsilyl group)

3.6 ppm (m) (18H methoxy group methyl group)

1.1 ppm (m) (12H dimethylphenylsilyl group methyl group)

From these results, it was confirmed that the obtained compound was a compound represented by the formula (i).

Moreover, the molecular occupying volumes of the main skeleton and side chain of the resulting compound, and the ratio (volume ratio) of the molecular occupying volume of the side chain to that of the main skeleton are shown in Table 2.

### Example 10 Synthesis of an Organic Silane Compound Represented by the Formula (j)

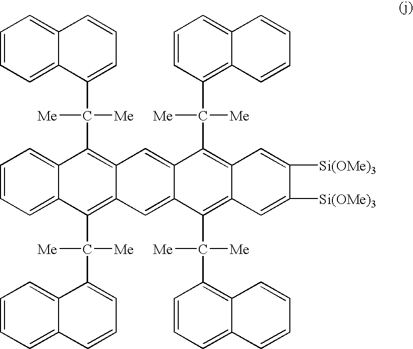

The inscribed compound was synthesized by the following method. First, 2,3,6,7-tetra(trimethylsilyl)-5,8-di(dimethylnaphthylalkyl)naphthalene was synthesized in the same method as in Reference Example of Example 7 except that Naphthalene-C(CH₃)₂NH was used in place of i-PrNH. The inscribed compound was obtained by applying the same method as in the synthetic example of Example 7 except that the above 2,3,6,7-tetra(trimethylsilyl)-5,8-di(dimethylnaphthylalkyl)naphthalene was used as the starting material and the sample obtained on the way of synthesis in place of 2,3,6,7-tetra(trimethylsilyl)-5,8-di(triisopropylsilyl) naphthalene.

The nuclear magnetic resonance (NMR) of the resulting compound was measured.

7.9 ppm (m) (6H pentacene)

7.8 ppm (m) (4H naphthalene)

7.6 ppm (m) (4H naphthalene)

7.5 ppm (m) (4H naphthalene)

7.4 ppm (m) (2H pentacene)

7.3 ppm (m) (8H naphthalene)

7.2 ppm (m) (4H naphthalene)

7.1 ppm (m) (4H naphthalene)

3.6 ppm (m) (18H methoxy group methyl group)

1.1 ppm (m) (12H dimethylphenylsilyl group methyl group)

From these results, it was confirmed that the obtained compound was a compound represented by the formula (j).

Moreover, the molecular occupying volumes of the main skeleton and side chain of the resulting compound, and the ratio (volume ratio) of the molecular occupying volume of the side chain to that of the main skeleton are shown in Table 2.

TABLE 2

| | Main skeleton (Å³) | Side chain (Å³) | Volume ratio (%) |
|---|---|---|---|
| Example 1 | 44 | 2.1 | 4.8 |
| Example 2 | 56 | 18.4 | 32.9 |
| Example 3 | 709 | 2.1 | 0.3 |
| Example 4 | 56 | 20.9 | 37.3 |
| Example 5 | 56 | 30.7 | 54.8 |
| Example 6 | 71 | 3.3 | 4.6 |
| Example 7 | 129 | 25 | 19.4 |
| Example 8 | 88 | 3.2 | 3.6 |
| Example 9 | 90 | 3.3 | 3.7 |
| Example 10 | 129 | 59.5 | 46.1 |

### Example 11 Formation of an Organic Thin Film Transistor

In order to manufacture an organic thin film transistor shown in FIG. 2, first, chromium was vapor-deposited on a substrate 1 made of silicon to form a gate electrode 2.

Then, a gate insulating film 3 made of a silicon nitride film was deposited by a plasma CVD method and then, chromium and gold were vapor-deposited in this order on the gate insulating film 3 to form source/drain electrodes (5, 6) by a usual lithographic technique.

In succession, the resulting substrate was dipped in a mixture solution of hydrogen peroxide and concentrated sulfuric acid (mixed ratio: 3:7) for one hour to carry out hydrophilic treatment of the surface of the gate insulating film 3. Thereafter, the obtained substrate was dipped in 20 mM solution prepared by dissolving 2′,6′,2″,6″-tetra-tert-butyl-[1,1′;4,4″;1″,1″′]quarter phenyltrimethoxysilane (compound of Example 1) in a nonaqueous solvent (for example, n-hexane) for 5 minutes in an anaerobic condition and slowly pulled up, followed by washing with a solvent to form an organic thin film 4, thereby forming an organic TFT.

The organic thin film transistor obtained above had a field effect mobility of 4.2×10⁻² cm²/Vs and an about six-digit ON/OFF ratio, exhibiting a good performance.

### Examples 12 to 20

The compounds shown in the following table were respectively applied to form a film in the same method as in Example 11 to form an organic thin film transistor. Each characteristic was evaluated with the result that a good performance as shown in the following table was obtained.

TABLE 3

| Example | Organic thin film material | Mobility | ON/OFF ratio |
|---|---|---|---|
| Example 12 | Compound of Example 2 | 5.0 × 10⁻² | 5 |
| Example 13 | Compound of Example 3 | 6.0 × 10⁻² | 5 |
| Example 14 | Compound of Example 4 | 7.5 × 10⁻² | 6 |
| Example 15 | Compound of Example 5 | 8.3 × 10⁻² | 6 |
| Example 16 | Compound of Example 6 | 7.2 × 10⁻² | 6 |
| Example 17 | Compound of Example 7 | 9.0 × 10⁻² | 6 |
| Example 18 | Compound of Example 8 | 9.2 × 10⁻² | 5 |
| Example 19 | Compound of Example 9 | 7.5 × 10⁻² | 6 |
| Example 20 | Compound of Example 10 | 8.3 × 10⁻² | 6 |

The invention thus described, it will be obvious that the same may be varied in many ways. Such variations are not to be regarded as a departure from the sprits and scope of the invention, and all such modifications as would be obvious to one skilled in the art are intended to be included within the scope of the following claims.

This application is related to Japanese applications Nos. 2004-243974 filed on Aug. 24, 2004 and 2004-243965 filed on Aug. 24, 2004, the disclosures of which are incorporated by reference in their entirety.

1. A side chain-containing type organic silane compound represented by the formula (I) R—SiX¹X²X³ wherein R represents a π-electron conjugate type organic residue composed of 3 to 10 units whose units are a group derived from a monocyclic aromatic hydrocarbon, a group derived from a monocyclic heterocyclic compound or the combination thereof, or an organic residue composed of 2 to 10 five-membered or six-membered rings, both of the residues having at least one side chain, and X¹, X² and X³, the same or different represent a group affording a hydroxyl group upon hydrolysis.

2. The side chain-containing type organic silane compound according to claim 1, wherein the R is an organic residue having a vinylene group between units.

3. The side chain-containing type organic silane compound according to claim 1, wherein the monocyclic aromatic hydrocarbon and monocyclic heterocyclic compound are benzene or thiophene.

4. The side chain-containing type organic silane compound according to claim 1, wherein a molecule volume occupied by the side chain is 60% or less of a molecule volume occupied by a main skeleton of the organic residue excluding the side chain.

5. The side chain-containing type organic silane compound according to claim 1, wherein the side chain is an alkyl group having 1 to 4 carbon atoms, trialkylsilyl group having 1 to 4 carbon atoms, di or triarylalkyl group containing an aryl group having 5 to 18 carbon atoms, or di or triarylsilyl group containing an aryl group having 5 to 18 carbon atoms.

6. The side chain-containing type organic silane compound according to claim 1, wherein the R has a linear symmetry with respect to a molecular axis.

7. The side chain-containing type organic silane compound according to claim 1, wherein the R has a point symmetry with respect to the center of the R as a whole.

8. The side chain-containing type organic silane compound according to claim 1, wherein the X¹, X² and X³ are the same and are a halogen atom or a lower alkoxy group.

9. The side chain-containing type organic silane compound according to claim 1, wherein the R is the organic residue of the condensed polycyclic compound and has an aryl group R2 between the R and Si.

10. The side chain-containing type organic silane compound according to claim 1, wherein the R has a following acene skeleton: 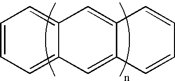
wherein n is 0 to 8.

11. A method of producing a side chain-containing type organic silane compound comprising reacting a compound represented by the formula (II) R—Li, wherein R represents a π-electron conjugate type organic residue composed of 3 to 10 units whose units are a group derived from a monocyclic aromatic hydrocarbon, a group derived from a monocyclic heterocyclic compound or the combination thereof, or an organic residue composed of 2 to 10 five-membered or six-membered rings, both of the residues having at least one side chain
with a compound represented by the formula (III) Y—SiX¹X²X³, wherein X¹, X² and X³, the same or different represent a group affording a hydroxyl group upon hydrolysis and Y represents a hydrogen atom, a halogen atom or a lower alkoxy group
to produce the side chain-containing type organic silane compound represented by the formula (I) R—SiX¹X¹X²X³, wherein R and X1 to X3 are as defined above.

12. A method of producing a side chain-containing type organic silane compound comprising reacting a compound represented by the formula (IV) R—MgX, wherein R represents a π-electron conjugate type organic residue composed of 3 to 10 units whose units are a group derived from a monocyclic aromatic hydrocarbon, a group derived from a monocyclic heterocyclic compound or the combination thereof, or an organic residue composed of 2 to 10 five-membered or six-membered rings, both of the residues having at least one side chain,
with a compound represented by the formula (III) Y—SiX¹X²X³, wherein X¹, X² and X³, the same or different represent a group affording a hydroxyl group upon hydrolysis and Y represents a hydrogen atom, a halogen atom or a lower alkoxy group
to produce the side chain-containing type organic silane compound represented by the formula (I) R—SiX¹X¹X²X³, wherein R and X1 to X3 are as defined above.

13. An organic thin film transistor comprising a substrate, an organic thin film, a gate electrode formed on one surface of the organic thin film through a gate insulating film and a source/drain electrode formed in contact with one surface or the other surface of the above organic thin film on both sides of the gate electrode, wherein the above organic thin film is a film derived from a side chain-containing type organic silane compound represented by the formula (I) R—SiX¹X²X³ wherein R represents a π-electron conjugate type organic residue composed of 3 to 10 units whose units are a group derived from a monocyclic aromatic hydrocarbon, a group derived from a monocyclic heterocyclic compound or the combination thereof, or an organic residue composed of 2 to 10 five-membered or six-membered rings, both of the residues having at least one side chain, the same or different represent a group affording a hydroxyl group upon hydrolysis.

14. A process of producing an organic thin film transistor comprising a substrate, an organic thin film, a gate electrode formed on one surface of the organic thin film through a gate insulating film and a source/drain electrode formed in contact with one surface or the other surface of the above organic thin film on both sides of the gate electrode, the process comprising a step of forming an organic thin film by laminating, as a monomolecular film or built-up film, a side chain-containing type organic silane compound represented by the formula (I) R—SiX¹X²X³ wherein R represents a π-electron conjugate type organic residue composed of 3 to 10 units whose units are a group derived from a monocyclic aromatic hydrocarbon, a group derived from a monocyclic heterocyclic compound or the combination thereof, or an organic residue composed of 2 to 10 five-membered or six-membered rings, both of the residues having at least one side chain, the residue having at least one side chain and X¹, X² and X³, the same or different represent a group affording a hydroxyl group upon hydrolysis.
